(19) United States
(12) Patent Application Publication
(10) Pub. No.: US 2006/0211025 A1
(43) Pub. Date: Sep. 21, 2006

(54) Molecular signatures of commonly fatal carcinomas

(75) Inventors: Andrew I. Su, San Diego, CA (US); Garret M. Hampton, San Diego, CA (US)

Correspondence Address: GENOMICS INSTITUTE OF THE;NOVARTIS RESEARCH FOUNDATION, 10675 JOHN JAY HOPKINS DRIVE, SUITE E225, SAN DIEGO, CA, 92121-1127, US

(73) Assignee: IRM LLC, Hamilton (BM)

(21) Appl. No.: 11/373,081

(22) Filed: Mar. 9, 2006

Related U.S. Application Data
(63) Continuation of application No. 10/167,755, filed on Jun. 10, 2002.
(60) Provisional application No. 60/297,277, filed on Jun. 10, 2001.

(51) Int. Cl. C12Q 1/68

(52) U.S. Cl. 435/6

(57) ABSTRACT

This invention provides methods, kits, and algorithms for obtaining molecular signatures of cells based on their gene expression profiles. Devices for carrying out molecular signature analysis of unknown samples are also provided.

### CROSS-REFERENCE TO RELATED APPLICATIONS

This application is a continuation of U.S. patent application Ser. No. 10/167,755 (filed Jun. 10, 2002) which claims the benefit of U.S. provisional application No. 60/297,277 (filed Jun. 10, 2001). The aforementioned applications are incorporated herein by reference in their entirety and for all purposes.

### BACKGROUND OF THE INVENTION

1. Field of the Invention

This invention pertains to the field of diagnosis, prognosis and treatment of carcinomas. In particular, the invention provides methods for identifying the anatomic origin of carcinomas.

2. Background

Cancer is a leading cause of death in the United States, causing one in four deaths, which is second only to heart disease. More than half a million people die of cancer each year in the United States. Four cancer sites, the lung, prostate, breast and colon, account for 56% of all new cancer cases and are the leading causes of cancer deaths for every racial and ethnic group, according to the Annual Report to the Nation on the Status of Cancer, 1973-1998 (see Howe et al., J. Nat'l. Cancer Institute, 93:824-842 (2001)).

In about 4% of all patients diagnosed with cancer, the observed tumor is due to metastasis and the primary tumor origin is undetermined (see Hillen, Postgrad. Med. J., 76:690-693 (2000)). Thus, a central goal of cancer biology is the identification of molecules or sets of molecules that are unique to specific human carcinomas, both for the development of diagnostics and drugs for the treatment of disease, as well as ultimately to understand the mechanistic basis of tissue-specific tumorigenesis. Thus, the identification of genes whose expression is uniquely characteristic of tumors of diverse anatomic origins remains a central challenge to the development of new cancer therapies (see St Croix et al., Science, 289:1197-1202 (2000); Bittner et al., Nature, 406:536-540 (2000); Perou et al., Nature, 406:747-752 (2000); and Golub et al., Science, 286:531-537 (1999)). The present invention fulfills this and other needs.

### SUMMARY OF THE INVENTION

The invention provides kits and methods for determining the origin of a tumor. In a first embodiment, the invention provides kits for identifying an origin of a tumor in a subject. These kits include: a) a probe that can detect an expression product of a gene in a first tumor class as indicated in Table 3; and b) a probe that can detect an expression product of a gene in a second tumor class as indicated in Table 3. The kits can also include additional probes, such two or more probes for each of the tumor classes, probes that are diagnostic for more than two tumor classes, or any combination thereof. In some embodiments, the kits include probes for at least one gene in each of at least ten tumor classes. The tumor classes for which the invention provides diagnostic kits include prostate cancer, breast cancer, colorectal cancer, lung adenocarcinoma, lung squamous cell carcinoma, ovarian cancer, gastroesophageal cancer, pancreatic cancer, liver cancer, kidney cancer and bladder cancer. The expression product that is detected can be, for example, an mRNA that is transcribed from the gene, a protein encoded by the gene, or a product of an enzymatic reaction catalyzed by a protein encoded by the gene.

Also provided by the invention are methods for identifying an origin of a tumor. These methods involve detecting in a tumor sample an expression level of at least two genes, each of which genes is diagnostic for a different tumor class as identified in Table 3. An elevated level expression for a gene indicates that the tumor originated from the tumor class for which the gene is diagnostic. The methods provided can be used to determine, for example, whether a tumor sample originated from a prostate cancer, breast cancer, colorectal cancer, lung adenocarcinoma, lung squamous cell carcinoma, ovarian cancer, gastroesophageal cancer, pancreatic cancer, liver cancer, kidney cancer or bladder cancer. In some embodiments, an expression level is determined for at least three genes, each of which genes is diagnostic for a different tumor class as identified in Table 3, for at least two genes that are both diagnostic for a single tumor class as identified in Table 3, or combinations thereof. For example, the invention provides methods in which an expression level is determined for at least two genes that are both diagnostic for a first tumor class as identified in Table 3, and at least two genes that are both diagnostic for a second tumor class as identified in Table 3. The expression level of a gene in the tumor sample can be compared to the expression level of the gene in a non-cancer control sample, or to the expression level of the gene in a control sample obtained from a tumor of a different tumor class.

The invention also provides methods for identifying an origin of a tumor by: a) providing a predictor set that comprises expression levels for two or more genes, each of which is diagnostic for a different tumor class as identified in Table 3; b) detecting in a tumor sample an expression level of at one gene that is diagnostic for a tumor class as identified in Table 3; and c) calculating a vector distance from the expression level obtained from the tumor sample to each of the expression levels of the predictor set. The shortest vector distance from the unknown sample to one of the members of the predictor set indicates the origin of the tumor. In some embodiments, the predictor set includes expression levels for at least three genes, each of which genes is diagnostic for a different tumor class as identified in Table 3. The predictor set can include expression levels for at least two genes that are both diagnostic for a single tumor class as identified in Table 3. In some embodiments, the predictor set includes expression levels for at least two genes that are both diagnostic for a first tumor class as identified in Table 3, and at least two genes that are both diagnostic for a second tumor class as identified in Table 3. The predictor set, in some embodiments, includes expression levels for one or more genes in each of at least ten tumor classes identified in Table 3.

Methods for obtaining a predictor set for classifying a sample into one of two or more classes are also provided by the invention. These methods involve: a) obtaining a value for one or more features for each of a plurality of members of each of the classes; b) determining a Wilcoxon rank score for each of the features to eliminate nonpredictive features; and c) ranking the remaining features by predictive accuracy using a support vector machine. In some embodiments, the features are genes and the values are expression levels of the genes. The classes into which the sample is to be classified can include, for example, tumor classes, disease states, exposure to different conditions, and the like. The invention also provides computer-readable media and computers that are programmed to carry out the methods for obtaining a predictor set. These methods can further involve classifying a sample into one of the classes by: a) determining a value for one or more features in the sample; and b) calculating a vector distance from the obtained for the feature in the sample to each of the expression levels of the predictor set, wherein the shortest vector distance indicates the class of which the sample is a member.

Methods for screening a subject for prostate cancer or at risk of developing prostate cancer are also provided by the invention. These methods involve:
a) detecting a level of expression of at least one gene in a sample of prostate tissue obtained from the subject to provide a first value, wherein the gene is selected from the group consisting of LIM, multidrug resistance-associated protein homolog (MRP4), T-cell receptor Ti rearranged gamma-chain, testican, AC005053 and cam kinase I; and
b) comparing the first value with a level of expression of the gene in a sample of prostate tissue obtained from a disease-free subject, wherein a greater expression level in the subject sample compared to the sample from the disease-free subject is indicative of the subject having prostate cancer or at risk of developing prostate cancer.

The invention also provides methods for screening a subject for ovarian cancer or at risk of developing ovarian cancer. These methods involve:
a) detecting a level of expression of at least one gene in a sample of ovarian tissue obtained from the subject to provide a first value, wherein the gene is selected from the group consisting of laminin, alpha 5; vacuolar proton pump, beta polypeptide; putative cytoskeletal protein, natriuretic peptide receptor A, eyes absent homolog, U90916, AL049313, S100 alpha, keratinocyte transglutaminase, GPCR64, meis1, spondin 1, GPCR39, AL050069, mammoglobin 2, and branched chain aminotransferase 1, cytosolic; mesothelin and kallikrein 6; and
b) comparing the first value with a level of expression of the gene in a sample of ovarian tissue obtained from a disease-free subject, wherein a greater expression level in the subject sample compared to the sample from the disease-free subject is indicative of the subject having ovarian cancer or at risk of developing ovarian cancer.

The invention also provides methods for monitoring the progression of prostate cancer in a subject having, or at risk of having a prostate cancer. These methods involve measuring a level of expression of at least one gene selected from the group consisting of LIM, multidrug resistance-associated protein homolog (MRP4), T-cell receptor Ti rearranged gamma-chain, testican, AC005053 and cam kinase I, in a prostate tissue sample obtained from the subject, wherein an increase in the level of expression of the gene over time is indicative of the progression of the prostate cancer in the tissue.

Also provided by the invention are methods for monitoring the progression of ovarian cancer in a subject having, or at risk of having, an ovarian cancer. These methods involve measuring a level of expression of at least one gene selected from the group consisting of laminin, alpha 5; vacuolar proton pump, beta polypeptide; putative cytoskeletal protein, natriuretic peptide receptor A, eyes absent homolog, U90916, AL049313, S100 alpha, keratinocyte transglutaminase, GPCR64, meis1, spondin 1, GPCR39, AL050069, mammoglobin 2, and branched chain aminotransferase 1, cytosolic, in an ovarian tissue sample obtained from the subject, wherein an increase in the level of expression of the gene over time is indicative of the progression of the ovarian cancer in the tissue.

The invention provides methods for identifying agents for use in treatment of prostate cancer comprising. These methods involve:
a) contacting a sample of diseased prostate cells with a candidate agent;
b) detecting a level of expression of at least one gene in the diseased prostate cells, wherein the gene is selected from the group consisting of LIM, multidrug resistance-associated protein homolog (MRP4), T-cell receptor Ti rearranged gamma-chain, testican, AC005053 and cam kinase I; and
c) comparing the level of expression of the gene in the sample in the presence of the candidate agent with a level of expression of the gene in cells that are not contacted with the candidate agent, wherein a decreased level of expression of the gene in the sample in the presence of the candidate agent relative to the expression of the gene in the sample in the absence of the candidate agent is indicative of an agent useful in the treatment of prostate cancer.

### DETAILED DESCRIPTION

Assays and Kits for Classifying Cell Types Based on Molecular Signatures

This invention provides devices, kits, methods and algorithms for classifying cell types on the basis of their “molecular signatures”, such as gene expression profiles. The methods and algorithms are useful, for example, for analyzing the effect of drugs, toxicants or other factors on cells. The present invention relates to the identification of genes that exhibit a characteristic pattern of expression in cells of a particular type or cells that are exposed to a type of stimulus. For example, the invention provides devices and methods by which one can identify the anatomic origin of the ten cancers that are most commonly fatal in the United States (prostate, breast, colorectum, lung, ovary, gastroesophagus, pancreas, liver, kidney and bladder). Subsets of genes whose expression is uniquely characteristic for these carcinomas are identified and used to develop the algorithms and methods of the invention. These algorithms are applied to a mRNA profile or protein expression data from an unknown tumor to determine the type of carcinoma. Such information is key to devising an appropriate treatment strategy. This aspect of the invention is also useful for studies of the mechanistic basis of tumorigenesis, and also finds application in the testing of potential anti-cancer therapeutic agents, and in the diagnosis and prognosis of cancer.

Thus, in one aspect, the invention provides molecular signatures of the ten most commonly fatal types of cancer. The genes that are expressed in the cancer types include those listed in Table 3. By virtue of their distinctive expression profiles, these genes can be utilized in the diagnosis, management, treatment and/or post-treatment follow-up of persons at risk for, with, or at risk for recurrence of cancers.

The algorithms and methods of the invention are useful not only for characterizing tumor cells, but also for characterizing other cells that exhibit differential expression of particular genes compared to other cells. For example, a cell that is obtained from an organism that has been exposed to a drug or toxin will generally exhibit differences in expression of one or more genes. By applying the algorithms of the invention, one can determine which of these differences are most predictive of exposure to the drug or toxin. These algorithms for obtaining a molecular signature of gene expression are generally applicable to any cell type.

Once a molecular signature is obtained, it can be used to analyze cells from a wide variety of samples. For example, a tissue sample can be obtained from the subject, a human or animal model, by known surgical methods, e.g., surgical resection or needle biopsy. A sample of bodily fluid, preferably blood, can also be obtained by standard methods. Plant cells can also be analyzed, as can cells of fungi and microorganisms, including prokaryotes.

As stated above, the molecular signatures found for particular carcinomas are particularly useful in identifying the anatomic origin, i.e., the tissue origin, of tumors present in a subject. The tissue origin of the tumor found in a subject, e.g., an animal, preferably a human, is of prostate, breast, colorectal, lung, ovarian, gastroesophageal, pancreatic, liver, kidney, or bladder tissue origin.

In a particularly useful embodiment, a method for identifying a tissue origin of a tumor in a subject comprises:
a) obtaining a sample of the tumor from the subject;
b) detecting a level of expression of at least one gene in each gene set designated for each cancer type as identified in Table 3, in the subject sample to provide a first value; and
c) comparing the first value with a level of expression of the gene in each gene set designated for each cancer type as identified in Table 3, in a sample obtained from a subject of each cancer type, wherein a greater level of expression of the gene in one gene set in the subject sample compared with the level of expression of the gene in each cancer type sample indicates the tissue origin of the tumor.

The tumor present in the subject can be a metastatic lesion or a primary tumor whose cellular features of tissue origin are not readily identifiable.

The cancer types identified in Table 3 include prostate (PR), bladder (BL), breast (BR), colorectal (CO), gastroesophageal (GA), kidney (KI), liver (LI), ovary (OV), pancreatic (PA), lung adenoma (LA), and lung squamous (LS) cancer types. Typically, the level of expression of the gene from the gene set designated for the particular cancer type is about 2-, 5-, 10- or 100-fold or more than the expression level of that gene in the other cancer types.

A sample of tumor can be taken from the subject by methods well known in the art such as a biopsy. A sample obtained from a subject of each of the cancer types identified in Table 3 can be obtained from different individuals having a specific cancer type, or can be a pre-established control for which expression of the gene in each gene set selected for each cancer type was determined at an earlier time.

In some embodiments of this method, it is desirable to determine the level of 2, 3, 5, 10 or more of the genes in each gene set designated for each cancer type as identified in Table 3.

The level of expression of at least one of the genes that make up the molecular signature in the samples obtained from the subject can be detected by measuring either the level of mRNA corresponding to the gene or the protein encoded by the gene. RNA can be isolated from the samples by methods well-known to those skilled in the art as described, e.g., in Ausubel et al., Current Protocols in Molecular Biology, 1:4.1.1-4.2.9 and 4.5.1-4.5.3, John Wiley & Sons, Inc. (1996). Methods for detecting the level of expression of mRNA are well-known in the art and include, but are not limited to, northern blotting, reverse transcription PCR, real time quantitative PCR and other hybridization methods.

A particularly useful method for detecting the level of mRNA transcripts expressed from a plurality of the disclosed genes involves hybridization of labeled mRNA to an ordered array of oligonucleotides. Such a method allows the level of transcription of a plurality of these genes to be determined simultaneously to generate gene expression profiles or patterns.

The oligonucleotides utilized in this hybridization method are typically bound to a solid support. Examples of solid supports include, but are not limited to, membranes, filters, slides, paper, nylon, wafers, fibers, magnetic or nonmagnetic beads, gels, tubing, polymers, polyvinyl chloride dishes, etc. Any solid surface to which the oligonucleotides can be bound, either directly or indirectly, either covalently or non-covalently, can be used. A particularly preferred solid substrate is a high-density array or DNA chip. These high-density arrays contain a particular oligonucleotide probe in a preselected location on the array. Each preselected location can contain more than one molecule of the particular probe. Because the oligonucleotides are at specified locations on the substrate, the hybridization patterns and intensities (which together result in a unique expression profile or pattern) can be interpreted in terms of expression levels of particular genes.

The oligonucleotide probes are preferably of sufficient length to specifically hybridize only to complementary transcripts of the above identified gene(s) of interest. As used herein, the term “oligonucleotide” refers to a single-stranded nucleic acid. Generally the oligonucleotides probes will be at least 16-20 nucleotides in length, although in some cases longer probes of at least 20-25 nucleotides will be desirable.

Once the probes are contacted with mRNA (or a cDNA copy) obtained from the, the presence of hybridized mRNA or cDNA from the sample is detected by methods known to those of skill in the art. For example, oligonucleotide probes can be labeled with one or more labeling moieties to permit detection of the hybridized probe/target polynucleotide complexes. Label moieties can include compositions that can be detected by spectroscopic, biochemical, photochemical, bioelectronic, immunochemical, electrical optical or chemical means. Examples of labeling moieties include, but are not limited to, radioisotopes, e.g., ³²P, ³³P, ³⁵S, chemiluminescent compounds, labeled-binding proteins, heavy metal atoms, spectroscopic markers, such as fluorescent markers and dyes, linked enzymes, mass spectrometry tags and magnetic labels.

Oligonucleotide probe arrays for expression monitoring can be prepared and used according to techniques which are well-known to those skilled in the art as described, e.g., in Lockhart et al., Nature Biotechnol., 14:1675-1680 (1996); McGall et al., Proc. Nat'l. Acad. Sci. USA, 93:13555-13460 (1996); and U.S. Pat. No. 6,040,138. Such DNA chips are commercially available from, for example, Affymetrix (Santa Clara, Calif.).

One can also detect expression of a protein encoded by one or more of the gene(s) that comprise the molecular signature. This can be accomplished by well-established methods, such as, for example, use of a probe that is detectably-labeled, or which can be subsequently-labeled. Generally, the probe is an antibody which recognizes the expressed protein. As used herein, the term antibody includes, but is not limited to, polyclonal antibodies, monoclonal antibodies, humanized or chimeric antibodies and biologically functional antibody fragments which are those fragments sufficient for binding of the antibody fragment to the protein.

For the production of antibodies to a protein encoded by one of the disclosed genes or to a fragment of the protein, various host animals may be immunized by injection with the polypeptide, or a portion thereof. Such host animals may include, but are not limited to, rabbits, mice and rats, to name but a few. Various adjuvants may be used to increase the immunological response, depending on the host species, including, but not limited to, Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum.

Polyclonal antibodies are heterogeneous populations of antibody molecules derived from the sera of animals immunized with an antigen, such as target gene product, or an antigenic functional derivative thereof. For the production of polyclonal antibodies, host animals, such as those described above, may be immunized by injection with the encoded protein, or a portion thereof, supplemented with adjuvants as also described above.

Monoclonal antibodies (mAbs), which are homogeneous populations of antibodies to a particular antigen, may be obtained by any technique which provides for the production of antibody molecules by continuous cell lines in culture. These include, but are not limited to, the hybridoma technique of Kohler and Milstein (Nature, Vol. 256, pp. 495-497 (1975); and U.S. Pat. No. 4,376,110), the human B-cell hybridoma technique (Kosbor et al., Immunology Today, Vol. 4, p. 72 (1983); Cole et al., Proc. Natl. Acad. Sci. USA, Vol. 80, pp. 2026-2030 (1983)), and the EBV-hybridoma technique (Cole et al., Monoclonal Antibodies and Cancer Therapy, Alan R. Liss, Inc., pp. 77-96 (1985)). Such antibodies may be of any immunoglobulin class, including IgG, IgM, IgE, IgA, IgD, and any subclass thereof. The hybridoma producing the mAb of this invention may be cultivated in vitro or in vivo. Production of high titers of mabs in vivo makes this the presently preferred method of production.

In addition, techniques developed for the production of “chimeric antibodies” (Morrison et al., Proc. Natl. Acad. Sci. USA, Vol. 81, pp. 6851-6855 (1984); Neuberger et al., Nature, Vol. 312, pp. 604-608 (1984); Takeda et al., Nature, Vol. 314, pp. 452-454 (1985)) by splicing the genes from a mouse antibody molecule of appropriate antigen specificity, together with genes from a human antibody molecule of appropriate biological activity, can be used. A chimeric antibody is a molecule in which different portions are derived from different animal species, such as those having a variable or hypervariable region derived from a murine mAb and a human immunoglobulin constant region.

Alternatively, techniques described for the production of single-chain antibodies (U.S. Pat. No. 4,946,778; Bird, Science, Vol. 242, pp. 423-426 (1988); Huston et al., Proc. Natl. Acad. Sci. USA, Vol. 85, pp. 5879-5883 (1988); and Ward et al., Nature, Vol. 334, pp. 544-546 (1989)) can be adapted to produce differentially expressed gene-single chain antibodies. Single chain antibodies are formed by linking the heavy and light chain fragments of the Fv region via an amino acid bridge, resulting in a single-chain polypeptide.

Most preferably, techniques useful for the production of “humanized antibodies” can be adapted to produce antibodies to the proteins, fragments or derivatives thereof. Such techniques are disclosed in U.S. Pat. Nos. 5,932,448; 5,693,762; 5,693,761; 5,585,089; 5,530,101; 5,569,825; 5,625,126; 5,633,425; 5,789,650; 5,661,016; and 5,770,429.

Antibody fragments which recognize specific epitopes may be generated by known techniques. For example, such fragments include, but are not limited to, the F(ab′)₂ fragments, which can be produced by pepsin digestion of the antibody molecule, and the Fab fragments, which can be generated by reducing the disulfide bridges of the F(ab′)₂ fragments. Alternatively, Fab expression libraries may be constructed (Huse et al., Science, Vol. 246, pp. 1275-1281 (1989)) to allow rapid and easy identification of monoclonal Fab fragments with the desired specificity.

The extent to which the known proteins are expressed in the sample is then determined by immunoassay methods which utilize the antibodies. Such immunoassay methods include, but are not limited to, dot blotting, western blotting, competitive and non-competitive protein binding assays, enzyme-linked immunosorbant assays (ELISA), immunohistochemistry, fluorescence-activated cell sorting (FACS), and others commonly used and widely-described in scientific and patent literature, and many employed commercially.

A particularly preferred immunoassay method for determining the level of expression of a large number of proteins that make up a molecular signature for a cell type is an antibody array. In this technique, antibodies, preferably monoclonal antibodies specific for the proteins of interest, are directly deposited at high density on a support, e.g., high density array. Similar technology has also been developed for preparing high density DNA microarrays. (See, e.g., Shalon et al., Genome Research, Vol. 6, pp. 639-645 (1996)). The antibody array is then incubated with a protein sample, e.g., a tumor sample from a subject as described above, which is prepared under conditions that reduce native protein-protein interactions. Following incubation, any unbound or non-specific binding proteins can be removed by washing. The proteins that are specifically bound to their respective antibodies on the array can then be detected. Since the antibodies are bound to the array in a predetermined order, the identity of the protein bound at each position can be ascertained. Measurement of the quantity of protein at all positions on the array thus reflects the protein expression pattern in the sample. The quantity of proteins bound to the array can be measured by several well known methods. For example, the proteins in the sample can be metabolically labeled with radioactive isotopes, e.g., ³⁵S for total proteins and ³²P for phosphorylated proteins. The amount of labeled proteins bound to each antibody on the array can be measured by autoradiography and densitometry. The protein sample can also be labeled by biotinylation in vitro. The biotinylated proteins bound on the array can then be detected by avidin or streptavidin which binds to biotin. If the avidin is conjugated with horseradish peroxidase or alkaline phosphatase, the bound protein can be visualized by enhanced chemical luminescence. The quantity of protein bound to each antibody indicates the level of that particular protein in the sample. Other methods can also be used to detect the proteins bound to the antibody array, e.g., immunochemical staining and matrix-assisted laser desorption/ionization-time of flight.

The invention also provides antibody-based panels for identifying the tissue origin, i.e., the anatomic site of origin, of a tumor in a subject. The panel comprises a set of antibody reagents, wherein the set includes at least one antibody reagent specific for detecting a protein encoded by at least one gene in each gene set designated for each cancer type as identified in Table 3. The tissue origin of the tumor is of prostate, breast, colorectal, lung, ovarian, gastroesophageal, pancreatic, liver, kidney, or bladder tissue origin. The term “antibody” is defined above and is preferably a monoclonal antibody specific for detecting the protein.

In some embodiments of the antibody-based panel, the set includes 2, 3, 5, 10 or more antibody reagents specific for detecting proteins encoded by 2, 3, 5, 10 or more genes, respectively, in each gene set designated for each cancer type as identified in Table 3.

The invention also provides devices for use in classifying cell types. For example, the invention provides DNA microarrays that include probes for two or more of the genes that make up an expression profile of a particular cell type. In presently preferred embodiments, each array will include probes that are diagnostic for two or more cell types. An array for characterizing cancers could include, for example, probes for some or all of the genes shown in Table 3 that are diagnostic of two or more of the indicated solid tumor types.

The invention also provides antibody arrays that include antibodies specific for at least one protein encoded by a gene in a gene set designated for each cancer type as identified in Table 3. Preferably the antibody arrays includes antibodies specific for 2, 3, 5, 10 or more proteins encoded by the respective genes in each gene set designated for each cancer type as set forth in Table 3.

A number of the genes in each gene set that distinguish one tumor type from another as identified in Table 3 are also found to be overexpressed in the different tumor types when compared to normal tissue. These tumor-specific genes can be utilized as biomarkers for the diagnosis, management, treatment and post-treatment of the various cancers described herein. For example, several genes that are tumor-specific for ovarian cancer and prostate cancer are described above, respectively. Identification of tumor-specific genes from the other gene sets designated for the cancer types including breast, colorectum, lung, ovary, gastroesophagus, pancreas, liver, kidney and bladder, can be readily determined by measuring the level of expression of the genes in a sample obtained from a each cancer type and comparing it to the level of expression of the genes in a sample obtained from the respective normal tissues. An increase in the level of expression of a gene(s) in the set of genes that classify the particular cancer type relative the the level of expression of the gene(s) in its respective normal tissue indicates that the gene(s) is a tumor-specific gene.

Accordingly, in one aspect, the invention also provides for diagnostic and prognostic assays which are capable of detecting differential expression of specific genes in ovarian and prostate cancers compared with normal ovarian and prostate tissues.

In one embodiment, a method for screening a subject for prostate cancer or at risk of developing prostate cancer is provided which comprises:
a) detecting a level of expression of at least one gene in a sample of prostate tissue obtained from the subject to provide a first value, wherein the gene is selected from the group consisting of LIM, multidrug resistance-associated protein homolog (MRP4), T-cell receptor Ti rearranged gamma-chain, testican, AC005053 and cam kinase I; and
b) comparing the first value with a level of expression of the gene in a sample of prostate tissue obtained from a disease-free subject, wherein a greater expression level in the subject sample compared to the sample from the disease-free subject is indicative of the subject having prostate cancer or at risk of developing prostate cancer.

In another embodiment, a method for screening a subject for ovarian cancer or at risk of developing ovarian cancer is provided which comprises:
a) detecting a level of expression of at least one gene in a sample of ovarian tissue obtained from the subject to provide a first value, wherein the gene is selected from the group consisting of laminin, alpha 5; vacuolar proton pump, beta polypeptide; putative cytoskeletal protein, natriuretic peptide receptor A, eyes absent homolog, U90916, AL049313, S100 alpha, keratinocyte transglutaminase, GPCR64, meis1, spondin 1, GPCR39, AL050069, mammoglobin 2, and branched chain aminotransferase 1, cytosolic; mesothelin and kallikrein 6.
b) comparing the first value with a level of expression of the gene in a sample of ovarian tissue obtained from a disease-free subject, wherein a greater expression level in the subject sample compared to the sample from the disease-free subject is indicative of the subject having ovarian cancer or at risk of developing ovarian cancer.

The prostate or ovarian tissue sample can be obtained from the subject, a human or animal model, by known surgical methods, e.g., surgical resection or needle biopsy. The sample taken from the disease-free subject can be a sample of normal prostate or ovarian tissue or bodily fluid from the same individual or from another individual. For example, in examination of a suspected prostate or ovarian cancer, the sample from the disease-free subject can be a sample of normal prostate or ovarian cells from the individual suspected of having prostate or ovarian cancer. These normal cells can be obtained from a site adjacent to the tissue suspected of containing the prostate or ovarian cells. Alternatively, the sample taken from the disease-free subject can be a sample of normal prostate or ovarian tissue obtained from another individual. The sample obtained from the disease-free subject can be obtained at the same time as the sample obtained from the subject, or can be a pre-established control for which expression of the gene was determined at an earlier time. The level of expression of the gene in the sample obtained from the disease-free subject is determined and quantitated using the same approach as used for the sample obtained from the subject.

The level of expression of at least one of the disclosed genes in the samples obtained from the subject and disease-free subject can be detected by measuring either the level of mRNA corresponding to the gene, the protein encoded by the gene or a fragment of the protein by methods well known in the art as described above. In the methods of the invention, the level of expression of one of the disclosed genes in a diseased prostate or ovarian tissue preferably differs from the level of expression of the gene in a non-diseased tissue by a statistically significant amount. In presently preferred embodiments, at least about a 2-fold difference in expression levels is observed. In some embodiments, the expression levels of a gene differ by at least about 5-, 10- or 100-fold or more in the diseased tissue compared to the non-diseased tissue.

In preferred embodiment of these methods, the level of expression of two, three or more genes is detected.

The invention also provides for methods of monitoring the progression of a cancer, e.g., a prostate or ovarian cancer, in a subject by measuring a level of expression of mRNA corresponding to, or protein encoded by, at least one of the tumor-specific genes that are differentially expressed in the cancer, in a sample obtained from the subject over time, i.e., at various stages of the disease. An increase in the level of expression of the gene(s) over time is indicative of the progression of the cancer. The level of expression of the gene(s) can be detected by standard methods as described above.

Assays to Identify Agents that Modulate Expression

In another aspect, a cell-based assay based on one or more of the genes that make up a molecular signature can be used to identify agents that modify the expression of these genes. Such agents find use, for example, in the treatment of the condition (e.g., a particular type of cancer) for which the molecular signature is diagnostic. These methods typically involve: a) contacting a sample obtained from a subject suspected of having the condition of interest with a candidate agent; b) detecting a level of expression of at least one gene that comprises the molecular signature (e.g., for cancer, a gene identified in Table 3); and c) comparing the level of expression of the gene in the sample in the presence of the candidate agent with a level of expression of the gene in the sample in the absence of the candidate agent, wherein an increased or decreased level of expression in the sample in the presence of the agent relative to the level of expression in the absence of the agent is indicative of an agent that can modulate the expression of the gene. The level of expression of the gene can be detected by, for example, measuring the level of mRNA corresponding to or protein encoded by the gene as described above. In presently preferred embodiments, the expression of more than one gene in the molecular signature is monitored for modulation by the candidate agent.

As used herein, the term “candidate agent” refers to any molecule that is capable of decreasing the level of mRNA corresponding to or protein encoded by at least one of the genes that comprise a molecular signature. The candidate agents can be natural or synthetic molecules such as proteins or fragments thereof, small molecule inhibitors, nucleic acid molecules, e.g., antisense nucleotides, ribozymes, double-stranded RNAs, organic and inorganic compounds and the like.

In particular, the cell-based assay can be utilized to identify agents that inhibit or decrease the expression of one or more genes that are differentially expressed, i.e., overexpressed in diseased cells, i.e., cancer cells, compared to non-diseased cells. As stated above, genes that are overexpressed in cancer tissue relative to the respective normal tissue can be discerned by measuring the expression level of the gene in a sample of both tissues and comparing the expression levels obtained for both tissues. Examples of tumor-specific genes that are overexpressed in ovarian and prostate cancers, respectively, are described above. Other cancer cells include breast, colorectal, gastroesophageal, pancreatic, liver, kidney and bladder cells.

In one embodiment, a method for identifying agents for use in treatment of prostate cancer is provided which comprises:
a) contacting a sample of diseased prostate cells with a candidate agent;
b) detecting a level of expression of at least one gene in the diseased prostate cells, wherein the gene is selected from the group consisting of LIM, multidrug resistance-associated protein homolog (MRP4), T-cell receptor Ti rearranged gamma-chain, testican, AC005053 and cam kinase I; and
c) comparing the level of expression of the gene in the sample in the presence of the candidate agent with a level of expression of the gene in cells that are not contacted with the candidate agent, wherein a decreased level of expression of the gene in the sample in the presence of the candidate agent relative to the expression of the gene in the sample in the absence of the candidate agent is indicative of an agent useful in the treatment of prostate cancer.

In another embodiment, a method of identifying agents useful in the treatment of ovarian cancer is provided which comprises:
a) contacting a sample of diseased ovarian cells with a candidate agent;
b) detecting a level of expression of at least one gene in the diseased ovarian cells, wherein the gene is selected from the group consisting of laminin, alpha 5; vacuolar proton pump, beta polypeptide; putative cytoskeletal protein, natriuretic peptide receptor A, eyes absent homolog, U90916, AL049313, S100 alpha, keratinocyte transglutaminase, GPCR64, meis1, spondin 1, GPCR39, AL050069, mammoglobin 2, and branched chain aminotransferase 1, cytosolic; and
c) comparing the level of expression of the gene in the sample in the presence of the candidate agent with a level of expression of the gene in cells that are not contacted with the candidate agent, wherein a decreased level of expression of the gene in the sample in the presence of the candidate agent relative to the expression of the gene in the sample in the absence of the candidate agent is indicative of an agent useful in the treatment of ovarian cancer.

Cell-free assays can also be used to identify compounds which are capable of interacting with a protein encoded by one or more of the genes that make up the molecular signature, or with a binding partner of one of these encoded proteins, to alter the activity of the protein or its binding partner. Cell-free assays can also be used to identify compounds which modulate the interaction between the encoded protein and its binding partner, such as a target peptide. In one embodiment, cell-free assays for identifying such compounds comprise a reaction mixture containing a protein encoded by one of the molecular signature component genes and a test compound or a library of test compounds in the presence or absence of the binding partner, e.g., a biologically inactive target peptide, or a small molecule. Accordingly, one example of a cell-free method for identifying agents useful in the modulation of the underlying condition for which the molecular signature is characteristic involves contacting a protein or functional fragment thereof or the protein binding partner with a test compound or library of test compounds and detecting the formation of complexes. For detection purposes, the protein can be labeled with a specific marker and the test compound or library of test compounds labeled with a different marker. Interaction of a test compound with the protein or fragment thereof or the protein binding partner can then be detected by measuring the level of the two labels after incubation and washing steps. The presence of the two labels is indicative of an interaction.

Interaction between molecules can also be assessed by using real-time BIA (Biomolecular Interaction Analysis, Pharmacia Biosensor AB) which detects surface plasmon resonance, an optical phenomenon. Detection depends on changes in the mass concentration of mass macromolecules at the biospecific interface and does not require labeling of the molecules. In one useful embodiment, a library of test compounds can be immobilized on a sensor surface, e.g., a wall of a micro-flow cell. A solution containing the protein, functional fragment thereof, or the protein binding partner is then continuously circulated over the sensor surface. An alteration in the resonance angle as indicated on a signal recording, indicates the occurrence of an interaction. This technique is described in more detail in BIA Technology Handbook by Pharmacia.

Another embodiment of a cell-free assay involves: a) combining a protein encoded by the gene, the protein binding partner, and a test compound to form a reaction mixture; and b) detecting interaction of the protein and the protein binding partner in the presence and absence of the test compounds. A considerable change (potentiation or inhibition) in the interaction of the protein and binding partner in the presence of the test compound compared to the interaction in the absence of the test compound indicates a potential agonist (mimetic or potentiator) or antagonist (inhibitor) of the protein's activity for the test compound. The components of the assay can be combined simultaneously or the protein can be contacted with the test compound for a period of time, followed by the addition of the binding partner to the reaction mixture. The efficacy of the compound can be assessed by using various concentrations of the compound to generate dose response curves. A control assay can also be performed by quantitating the formation of the complex between the protein and its binding partner in the absence of the test compound.

Formation of a complex between the protein and its binding partner can be detected by using detectably-labeled proteins, such as radiolabeled, fluorescently-labeled or enzymatically-labeled protein or its binding partner, by immunoassay or by chromatographic detection.

In preferred embodiments, the protein or its binding partner can be immobilized to facilitate separation of complexes from uncomplexed forms of the protein and its binding partner and automation of the assay. Complexation of the protein to its binding partner can be achieved in any type of vessel, e.g., microtitre plates, microcentrifuge tubes and test tubes. In particularly preferred embodiment, the protein can be fused to another protein, e.g., glutathione-S-transferase to form a fusion protein which can be adsorbed onto a matrix, e.g., glutathione Sepharose® beads (Sigma Chemical, St. Louis, Mo.) which are then combined with the labeled protein partner, e.g., labeled with ³⁵S, and test compound and incubated under conditions sufficient to formation of complexes. Subsequently, the beads are washed to remove unbound label and the matrix is immobilized and the radiolabel is determined.

Another method for immobilizing proteins on matrices involves utilizing biotin and streptavidin. For example, the protein can be biotinylated using biotin N-hydroxy-succinimide (NHS) using well-known techniques and immobilized in the well of steptavidin-coated plates.

Cell-free assays can also be used to identify agents which are capable of interacting with a protein encoded by at least one gene that comprises a molecular signature and modulate the activity of the protein encoded by the gene. In one embodiment, the protein is incubated with a test compound and the catalytic activity of the protein is determined. In another embodiment, the binding affinity of the protein to a target molecule can be determined by methods known in the art.

The present invention also provides for both prophylactic and therapeutic methods of treating a subject having or at risk of having a disorder or condition for which a molecular signature is diagnostic. Subjects at risk for such disorders can be identified by a prognostic assay, e.g., as described above. Administration of a prophylactic agent can occur prior to the manifestation of symptoms characteristic of the disorder or condition, such that development of the disorder is prevented or delayed in its progression. With respect to treatment of the disorder, it is not required that the cell, e.g., cancer cell, be killed or induced to undergo cell death. Instead, all that is required to achieve treatment of the disorder is that the tumor growth be slowed down to some degree or that some of the abnormal cells revert back to normal. Examples of suitable therapeutic agents include, but are not limited to, antisense nucleotides, ribozymes, double-stranded RNAs and antagonists. The molecular signatures of the invention are useful for monitoring the efficacy of a particular course of treatment for the disorder or condition.

As used herein, the term “antisense” refers to nucleotide sequences that are complementary to a portion of an RNA expression product of at least one of the disclosed genes. “Complementary” nucleotide sequences refer to nucleotide sequences that are capable of base-pairing according to the standard Watson-Crick complementarity rules. That is, purines will base-pair with pyrimidine to form combinations of guanine:cytosine and adenine:thymine in the case of DNA, or adenine:uracil in the case of RNA. Other less common bases, e.g., inosine, 5-methylcytosine, 6-methyladenine, hypoxanthine and others may be included in the hybridizing sequences and will not interfere with pairing.

When introduced into a host cell, antisense nucleotide sequences specifically hybridize with the cellular mRNA and/or genomic DNA corresponding to the gene(s) so as to inhibit expression of the encoded protein, e.g., by inhibiting transcription and/or translation within the cell.

The isolated nucleic acid molecule comprising the antisense nucleotide sequence can be delivered, e.g., as an expression vector, which when transcribed in the cell, produces RNA which is complementary to at least a unique portion of the encoded mRNA of the gene(s). Alternatively, the isolated nucleic acid molecule comprising the antisense nucleotide sequence is an oligonucleotide probe which is prepared ex vivo and, which, when introduced into the cell, results in inhibiting expression of the encoded protein by hybridizing with the mRNA and/or genomic sequences of the gene(s).

Preferably, the oligonucleotide contains artificial internucleotide linkages which render the antisense molecule resistant to exonucleases and endonucleases, and thus are stable in the cell. Examples of modified nucleic acid molecules for use as antisense nucleotide sequences are phosphoramidate, phosporothioate and methylphosphonate analogs of DNA as described, e.g., in U.S. Pat. No. 5,176,996; 5,264,564; and 5,256,775. General approaches to preparing oligomers useful in antisense therapy are described, e.g., in Van der Krol, BioTechniques, Vol. 6, pp. 958-976 (1988); and Stein et al., Cancer Res., Vol. 48, pp. 2659-2668 (1988).

Typical antisense approaches, involve the preparation of oligonucleotides, either DNA or RNA, that are complementary to the encoded mRNA of the gene. The antisense oligonucleotides will hybridize to the encoded mRNA of the gene and prevent translation. The capacity of the antisense nucleotide sequence to hybridize with the desired gene will depend on the degree of complementarity and the length of the antisense nucleotide sequence. Typically, as the length of the hybridizing nucleic acid increases, the more base mismatches with an RNA it may contain and still form a stable duplex or triplex. One skilled in the art can determine a tolerable degree of mismatch by use of conventional procedures to determine the melting point of the hybridized complexes.

Antisense oligonucleotides are preferably designed to be complementary to the 5′ end of the mRNA, e.g., the 5′ untranslated sequence up to and including the regions complementary to the mRNA initiation site, i.e., AUG. However, oligonucleotide sequences that are complementary to the 3′ untranslated sequence of mRNA have also been shown to be effective at inhibiting translation of mRNAs as described, e.g., in Wagner, Nature, Vol. 372, pp. 333 (1994). While antisense oligonucleotides can be designed to be complementary to the mRNA coding regions, such oligonucleotides are less efficient inhibitors of translation.

Regardless of the mRNA region to which they hybridize, antisense oligonucleotides are generally from about 15 to about 25 nucleotides in length.

The antisense nucleotide can also comprise at least one modified base moiety, e.g., 3-methylcytosine, 5,-methylcytosine, 7-methylguanine, 5-fluorouracil, 5-bromouracil, and may also comprise at least one modified sugar moiety, e.g., arabinose, hexose, 2-fluorarabinose, and xylulose.

In another embodiment, the antisense nucleotide sequence is an alpha-anomeric nucleotide sequence. An alpha-anomeric nucleotide sequence forms specific double stranded hybrids with complementary RNA, in which, contrary to the usual beta-units, the strands run parallel to each other as described e.g., in Gautier et al., Nucl. Acids. Res., Vol. 15, pp. 6625-6641 (1987).

Antisense nucleotides can be delivered to cells which express the described genes in vivo by various techniques, e.g., injection directly into the prostate tissue site, entrapping the antisense nucleotide in a liposome, by administering modified antisense nucleotides which are targeted to the prostate cells by linking the antisense nucleotides to peptides or antibodies that specifically bind receptors or antigens expressed on the cell surface.

However, with the above-mentioned delivery methods, it may be difficult to attain intracellular concentrations sufficient to inhibit translation of endogenous mRNA. Accordingly, in a preferred embodiment, the nucleic acid comprising an antisense nucleotide sequence is placed under the transcriptional control of a promoter, i.e., a DNA sequence which is required to initiate transcription of the specific genes, to form an expression construct. The use of such a construct to transfect cells results in the transcription of sufficient amounts of single stranded RNAs to hybridize with the endogenous mRNAs of the described genes, thereby inhibiting translation of the encoded mRNA of the gene. For example, a vector can be introduced in vivo such that it is taken up by a cell and directs the transcription of the antisense nucleotide sequence. Such vectors can be constructed by standard recombinant technology methods. Typical expression vectors include bacterial plasmids or phage, such as those of the pUC or Bluescript.™ plasmid series, or viral vectors such as adenovirus, adeno-associated virus, herpes virus, vaccinia virus and retrovirus adapted for use in eukaryotic cells. Expression of the antisense nucleotide sequence can be achieved by any promoter known in the art to act in mammalian cells. Examples of such promoters include, but are not limited to, the promoter contained in the 3′ long terminal repeat of Rous sarcoma virus as described, e.g., in Yamamoto et al., Cell, Vol. 22, pp. 787-797 (1980); the herpes thymidine kinase promoter as described, e.g., in Wagner et al., Proc. Natl. Acad. Sci. USA, Vol. 78, pp. 1441-1445 (1981); the SV40 early promoter region as described, e.g., in Bemoist and Chambon, Nature, Vol. 290, pp. 304-310 (1981); and the regulatory sequences of the metallothionein gene as described, e.g., in Brinster et al., Nature, Vol. 296, pp. 39-42 (1982).

Ribozymes are RNA molecules that specifically cleave other single-stranded RNA in a manner similar to DNA restriction endonucleases. By modifying the nucleotide sequences encoding the RNAs, ribozymes can be synthesized to recognize specific nucleotide sequences in a molecule and cleave it as described, e.g., in Cech, J. Amer. Med. Assn., Vol. 260, p. 3030 (1988). Accordingly, only mRNAs with specific sequences are cleaved and inactivated.

Two basic types of ribozymes include the “hammerhead”-type as described for example in Rossie et al., Pharmac. Ther., Vol. 50, pp. 245-254 (1991); and the hairpin ribozyme as described, e.g., in Hampel et al., Nucl. Acids Res., Vol. 18, pp. 299-304 (1999) and U.S. Pat. No. 5,254,678. Intracellular expression of hammerhead and hairpin ribozymes targeted to mRNA corresponding to at least one of the disclosed genes can be utilized to inhibit protein encoded by the gene.

Ribozymes can either be delivered directly to cells, in the form of RNA oligonucleotides incorporating ribozyme sequences, or introduced into the cell as an expression vector encoding the desired ribozymal RNA. Ribozyme sequences can be modified in essentially the same manner as described for antisense nucleotides, e.g., the ribozyme sequence can comprise a modified base moiety.

Double-stranded RNA, i.e., sense-antisense RNA, corresponding to at least one of the disclosed genes, can also be utilized to interfere with expression of at least one of the disclosed genes. Interference with the function and expression of endogenous genes by double-stranded RNA has been shown in various organisms such as C. elegans as described, e.g., in Fire et al., Nature, Vol. 391, pp. 806-811 (1998); drosophilia as described, e.g., in Kennerdell et al., Cell, Vol. 95, No. 7, pp. 1017-1026 (1998); and mouse embryos as described, e.g., in Wianni et al., Nat. Cell Biol., Vol. 2, No. 2, pp. 70-75 (2000). Such double-stranded RNA can be synthesized by in vitro transcription of single-stranded RNA read from both directions of a template and in vitro annealing of sense and antisense RNA strands. Double-stranded RNA can also be synthesized from a cDNA vector construct in which the gene of interest is cloned in opposing orientations separated by an inverted repeat. Following cell transfection, the RNA is transcribed and the complementary strands reanneal. Double-stranded RNA corresponding to at least one of the disclosed genes could be introduced into a prostate cell by cell transfection of a construct such as that described above.

The term “antagonist” refers to a molecule which, when bound to the protein encoded by the gene, inhibits its activity. Antagonists can include, but are not limited to, peptides, proteins, carbohydrates, and small molecules.

In a particularly useful embodiment, the antagonist is an antibody specific for the protein expressed by the gene. Antibodies useful as therapeutics encompass the antibodies as described above. The antibody alone may act as an effector of therapy or it may recruit other cells to actually effect cell killing. The antibody may also be conjugated to a reagent such as a chemotherapeutic, radionuclide, ricin A chain, cholera toxin, pertussis toxin, etc., and serve as a target agent. Alternatively, the effector may be a lymphocyte carrying a surface molecule that interacts, either directly or indirectly, with a tumor target. Various effector cells include cytotoxic T cells and NK cells.

Examples of the antibody-therapeutic agent conjugates which can be used in therapy include, but are not limited to: 1) antibodies coupled to radionuclides, such as 125I, ¹³¹I, ¹²³I , ¹¹¹In, ¹⁰⁵Rh, ¹⁵³Sm, ⁶⁷Cu, ⁶⁷Ga, 166Ho, ¹⁷⁷Lu, ¹⁸⁶Re and ¹⁸⁸Re, and as described, e.g., in Goldenberg et al., Cancer Res., Vol. 41, pp. 4354-4360 (1981); Carrasquillo et al., Cancer Treat. Rep., Vol. 68, pp. 317-328 (1984); Zalcberg et al., J. Natl. Cancer Inst., Vol. 72, pp. 697-704 (1984); Jones et al., Int. J. Cancer, Vol. 35, pp. 715-720 (1985); Lange et al., Surgery, Vol. 98, pp. 143-150 (1985); Kaltovich et al., J. Nucl. Med., Vol. 27, p. 897 (1986); Order et al., Int. J. Radiother. Oncol. Biol. Phys., Vol. 8, pp. 259-261 (1982); Courtenay-Luck et al., Lancet, Vol. 1, pp. 1441-1443 (1984); and Ettinger et al., Cancer Treat. Rep., Vol. 66, pp. 289-297 (1982); (2) antibodies coupled to drugs or biological response modifiers such as methotrexate, adriamycin, and lymphokines such as interferon as described, for, e.g., in Chabner et al., Cancer, Principles and Practice of Oncology, Philadelphia, Pa., J. B. Lippincott Co. Vol. 1, pp. 290-328 (1985); Oldham et al., Cancer, Principles and Practice of Oncology, Philadelphia, Pa., J. B. Lippincott Co., Vol. 2, pp. 2223-2245 (1985); Deguchi et al., Cancer Res., Vol. 46, pp. 3751-3755 (1986); Deguchi et al., Fed. Proc., Vol. 44, p. 1684 (1985); Embleton et al., Br. J. Cancer, Vol. 49, pp. 559-565 (1984); and Pimm et al., Cancer Immunol. Immunother., Vol. 12, pp. 125-134 (1982); (3) antibodies coupled to toxins, as described, for example, in Uhr et al., Monoclonal Antibodies and Cancer, Academic Press, Inc., pp. 85-98 (1983); Vitetta et al., Biotechnology and Bio. Frontiers, Ed. P. H. Abelson, pp. 73-85 (1984); and Vitetta et al., Science, Vol. 219, pp. 644-650 (1983); (4) heterofunctional antibodies, for example, antibodies coupled or combined with another antibody so that the complex binds both to the carcinoma and effector cells, e.g., killer cells such as T cells, as described, for example, in Perez et al., J. Exper. Med., Vol. 163, pp. 166-178 (1986); and Lau et al., Proc. Natl. Acad. Sci. USA, Vol. 82, pp. 8648-8652 (1985); and (5) native, i.e., non-conjugated or non-complexed antibodies, as described in, for example, Herlyn et al., Proc. Natl. Acad. Sci. USA, Vol. 79, pp. 4761-4765 (1982); Schulz et al., Proc. Natl. Acad. Sci. USA, Vol. 80, pp. 5407-5411 (1983); Capone et al., Proc. Natl. Acad. Sci. USA, Vol. 80, pp. 7328-7332 (1983); Sears et al., Cancer Res., Vol. 45, pp. 5910-5913 (1985); Nepom et al., Proc. Natl. Acad. Sci. USA, Vol. 81, pp. 2864-2867 (1984); Koprowski et al., Proc. Natl. Acad. Sci. USA, Vol. 81, pp. 216-219 (1984); and Houghton et al., Proc. Natl. Acad. Sci. USA, Vol. 82, pp. 1242-1246 (1985).

Methods for coupling an antibody or fragment thereof to a therapeutic agent as described above are well known in the art and are described, e.g., in the methods provided in the references above. In yet another embodiment, the antagonist useful as a therapeutic for treating cancer, e.g., prostate cancer, can be an inhibitor of a protein encoded by one of the disclosed genes.

In the case of treatment with an antisense nucleotide, the method comprises administering a therapeutically effective amount of an isolated nucleic acid molecule comprising an antisense nucleotide sequence derived from at least one gene identified in Table 3 or the ovarian and prostate tumor-specific genes disclosed herein. In one embodiment, the gene is preferably an ovarian tumor-specific gene selected from the group consisting of laminin, alpha 5; vacuolar proton pump, beta polypeptide; putative cytoskeletal protein, natriuretic peptide receptor A, eyes absent homolog, U90916, AL049313, S100 alpha, keratinocyte transglutaminase, GPCR64, meisl, spondin 1, GPCR39, AL050069, mammoglobin 2, and branched chain aminotransferase 1, cytosolic 4.

In another embodiment, the gene is preferably a prostate tumor-specific gene selected from the group consisting of LIM, multidrug resistance-associated protein homolog (MRP4), T-cell receptor Ti rearranged gamma-chain, testican, AC005053 and cam kinase I. The term “isolated” nucleic acid molecule means that the nucleic acid molecule is removed from its original environment (e.g., the natural environment if it is naturally occurring). For example, a naturally occurring nucleic acid molecule is not isolated, but the same nucleic acid molecule, separated from some or all of the co-existing materials in the natural system, is isolated, even if subsequently reintroduced into the natural system. Such nucleic acid molecules could be part of a vector or part of a composition and still be isolated, in that such vector or composition is not part of its natural environment.

With respect to treatment with a ribozyme or double-stranded RNA molecule, the method comprises administering a therapeutically effective amount of a nucleotide sequence encoding a ribozyme, or a double-stranded RNA molecule, wherein the nucleotide sequence encoding the ribozyme/double-stranded RNA molecule has the ability to decrease the transcription/translation of at least one gene identified in Table 3 or the ovarian and tumor-specific genes disclosed herein, respectively.

In the case of treatment with an antagonist, the method comprises administering to a subject a therapeutically effective amount of an antagonist that inhibits a protein encoded by at least one gene identified in Table 3 or the ovarian and prostate tumor-specific genes disclosed herein.

A “therapeutically effective amount” of an isolated nucleic acid molecule comprising an antisense nucleotide, nucleotide sequence encoding a ribozyme, double-stranded RNA, or antagonist, refers to a sufficient amount of one of these therapeutic agents to treat a cancer, e.g., a prostate cancer (e.g., to limit prostate tumor growth or to slow or block tumor metastasis). The determination of a therapeutically effective amount is well within the capability of those skilled in the art. For any therapeutic, the therapeutically effective dose can be estimated initially either in cell culture assays, e.g., of neoplastic cells, or in animal models, usually mice, rabbits, dogs, or pigs. The animal model may also be used to determine the appropriate concentration range and route of administration. Such information can then be used to determine useful doses and routes for administration in humans.

Therapeutic efficacy and toxicity may be determined by standard pharmaceutical procedures in cell cultures or experimental animals, e.g., ED50 (the dose therapeutically effective in 50% of the population) and LD50 (the dose lethal to 50% of the population). The dose ratio between toxic and therapeutic effects is the therapeutic index, and it can be expressed as the ratio, LD50/ED50. Antisense nucleotides, ribozymes, double-stranded RNAs, and antagonists which exhibit large therapeutic indices are preferred. The data obtained from cell culture assays and animal studies is used in formulating a range of dosage for human use. The dosage contained in such compositions is preferably within a range of circulating concentrations that include the ED50 with little or no toxicity. The dosage varies within this range, depending upon the dosage form employed, sensitivity of the patient, and the route of administration.

The exact dosage will be determined by the practitioner, in light of factors related to the subject that requires treatment. Dosage and administration are adjusted to provide sufficient levels of the active moiety or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy.

Normal dosage amounts may vary from 0.1 to 100,000 micrograms, up to a total dose of about 1 g, depending upon the route of administration. Guidance as to particular dosages and methods of delivery is provided in the literature and generally available to practitioners in the art. Those skilled in the art will employ different formulations for nucleotides than for antagonists.

For therapeutic applications, the antisense nucleotides, nucleotide sequences encoding ribozymes, double-stranded RNAs (whether entrapped in a liposome or contained in a viral vector) and antagonists are preferably administered as pharmaceutical compositions containing the therapeutic agent in combination with one or more pharmaceutically acceptable carriers. The compositions may be administered alone or in combination with at least one other agent, such as stabilizing compound, which may be administered in any sterile, biocompatible pharmaceutical carrier, including, but not limited to, saline, buffered saline, dextrose, and water. The compositions may be administered to a patient alone, or in combination with other agents, drugs or hormones.

The pharmaceutical compositions may be administered by any number of routes, including, but not limited to, oral, intravenous, intramuscular, intra-articular, intra-arterial, intramedullary, intrathecal, intraventricular, transdermal, subcutaneous, intraperitoneal, intranasal, enteral, topical, sublingual, or rectal means.

In addition to the active ingredients, these pharmaceutical compositions may contain suitable pharmaceutically-acceptable carriers comprising excipients and auxiliaries which facilitate processing of the active compounds into preparations which can be used pharmaceutically. Further details on techniques for formulation and administration may be found in the latest edition of Remington's Pharmaceutical Sciences (Maack Publishing Co., Easton, Pa.).

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient.

Pharmaceutical preparations for oral use can be obtained through combination of active compounds with solid excipient, optionally grinding a resulting mixture, and processing the mixture of granules, after adding suitable auxiliaries, if desired, to obtain tablets or dragee cores. Suitable excipients are carbohydrate or protein fillers, such as sugars, including lactose, sucrose, mannitol, or sorbitol; starch from corn, wheat, rice, potato, or other plants; cellulose, such as methyl cellulose, hydroxypropylmethyl-cellulose, or sodium carboxymethylcellulose; gums including arabic and tragacanth; and proteins such as gelatin and collagen. If desired, disintegrating or solubilizing agents may be added, such as the cross-linked polyvinyl pyrrolidone, agar, alginic acid, or a salt thereof, such as sodium alginate.

Dragee cores may be used in conjunction with suitable coatings, such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound, i.e., dosage.

Pharmaceutical preparations which can be used orally include push-fit capsules made of gelatin, as well as soft, sealed capsules made of gelatin and a coating, such as glycerol or sorbitol. Push-fit capsules can contain active ingredients mixed with a filler or binders, such as lactose or starches, lubricants, such as talc or magnesium stearate, and, optionally, stabilizers. In soft capsules, the active compounds may be dissolved or suspended in suitable liquids, such as fatty oils, liquid, or liquid polyethylene glycol with or without stabilizers.

Pharmaceutical formulations suitable for parenteral administration may be formulated from aqueous solutions, preferably in physiologically compatible buffers such as Hanks' solution, Ringer's solution, or physiologically buffered saline. Aqueous injection suspensions may contain substances which increase the viscosity of the suspension, such as sodium carboxymethyl cellulose, sorbitol, or dextran. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Non-lipid polycationic amino polymers may also be used for delivery. Optionally, the suspension may also contain suitable stabilizers or agents which increase the solubility of the compounds to allow for the preparation of highly concentrated solutions.

For topical or nasal administration, penetrants appropriate to the particular barrier to be permeated are used in the formulation. Such penetrants are generally known in the art.

The pharmaceutical compositions of the present invention may be manufactured in a manner that is known in the art, e.g., by means of conventional mixing, dissolving, granulating, dragee-making, levigating, emulsifying, encapsulating, entrapping, or lyophilizing processes.

The pharmaceutical composition may be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding free base forms. In other cases, the preferred preparation may be a lyophilized powder which may contain any or all of the following: 1-50 mM histidine, 0.1-2% sucrose, and 2-7% mannitol, at a pH range of 4.5 to 5.5, that is combined with buffer prior to use.

After pharmaceutical compositions have been prepared, they can be placed in an appropriate container and labeled for treatment of an indicated condition. For administration of the antisense nucleotide or antagonist, such labeling would include amount, frequency, and method of administration. Those skilled in the art will employ different formulations for antisense nucleotides than for antagonists, e.g., antibodies or inhibitors. Pharmaceutical formulations suitable for oral administration of proteins are described, e.g., in U.S. Pat. Nos. 5,008,114; 5,505,962; 5,641,515; 5,681,811; 5,700,486; 5,766,633; 5,792,451; 5,853,748; 5,972,387; 5,976,569; and 6,051,561.

In another aspect, the treatment of a subject with a therapeutic agent, such as those described above, can be monitored by detecting the level of expression of mRNA or protein encoded by at least one of the disclosed genes identified in Table 3 or the ovarian and prostate tumor-specific genes disclosed herein. These measurements will indicate whether the treatment is effective or whether it should be adjusted or optimized. Accordingly, one or more of the genes described herein can be used as a marker for the efficacy of a drug during clinical trials.

In a particularly useful embodiment, a method for monitoring the efficacy of a treatment of a subject having a prostate or ovarian cancer, or at risk of having such a cancer, with an agent (e.g., an antagonist, protein, nucleic acid, small molecule, or other therapeutic agent or candidate agent identified by the screening assays described herein) is provided comprising:
a) obtaining a pre-administration sample from a subject prior to administration of the agent,
b) detecting the level of expression of mRNA corresponding to, or protein encoded by the gene, or activity of the protein encoded by the gene identified in Table 3, or the ovarian and prostate tumor-specific genes disclosed herein, respectively, in the pre-administration sample;
c) obtaining one or more post-administration samples from the subject,
d) detecting the level of expression of mRNA corresponding to, or protein encoded by the gene, or activity of the protein encoded by the gene in the post-administration sample or samples,
e) comparing the level of expression of mRNA or protein encoded by the gene, or activity of the protein encoded by the gene in the pre-administration sample with the level of expression of mRNA or protein encoded by the gene, or activity of the protein encoded by the gene in the post-administration sample or samples, and
f) adjusting the administration of the agent accordingly.

For example, increased administration of the agent may be desirable to decrease the level of expression or activity of the gene to lower levels than detected, i.e., to increase the effectiveness of the agent. Alternatively, decreased administration of the agent may be desirable to increase expression or activity of the gene to higher levels than detected, i.e., to decrease the effectiveness of the agent.

In another aspect, a method for inhibiting undesired proliferation of a cancer cell, particularly a prostate or ovarian cell is provided which utilizes a therapeutic agent as described above, e.g., an antisense nucleotide, a ribozyme, a double-stranded RNA, and an antagonist such as an antibody. Preferably, the prostate or ovarian cell is present in a human. The undesired proliferation of the prostate or ovarian cell is associated with a condition including, but not limited to localized prostate cancer, metastatic prostate cancer, benign prostatic hyperplasia and ovarian cancer.

With respect to inhibition of proliferation of a prostate or ovarian cell utilizing an antisense nucleotide, the method comprises administering to the prostate or ovarian cell a therapeutically effective amount of an isolated nucleic acid molecule comprising an antisense nucleotide sequence derived from at least one gene identified in Tables 3 or the ovarian and prostate tumor-specific genes disclosed herein, respectively, wherein the antisense nucleotide has the ability to decrease the transcription/translation of the gene.

With respect to inhibition of proliferation of a prostate or ovarian cell utilizing a ribozyme, such a method comprises administering to the prostate or ovarian cell a therapeutically effective amount of a nucleotide sequence encoding the ribozyme, which has the ability to decrease the transcription/translation of at least one gene identified in Table 3, or the ovarian and prostate tumor-specific genes disclosed herein, respectively.

With respect to inhibition of proliferation of a prostate or ovarian cell utilizing a double-stranded RNA, the method comprises administering to the prostate cell a therapeutically effective amount of a double-stranded RNA corresponding to at least one gene identified in Table 3 or the ovarian and prostate tumor-specific genes disclosed herein.

With respect to inhibition of proliferation of a prostate cell utilizing an antagonist, the method comprises administering to the prostate or ovarian cell a therapeutically effective amount of an antagonist that inhibits a protein encoded by at least one gene identified in Table 3 or the ovarian and prostate tumor-specific genes disclosed herein.

In the context of inhibiting undesired proliferation of a cancer cell such as prostate or ovarian cell, a “therapeutically effective amount” of an isolated nucleic acid molecule comprising an antisense nucleotide, a nucleotide sequence encoding a ribozyme, a double-stranded RNA, or antagonist, refers to a sufficient amount of one of these therapeutic agents to inhibit proliferation of a cancer cell (e.g., to inhibit or stabilize cellular growth of the cancer cell) and can be determined as described above.

In another aspect, a viral vector is provided which comprises a promoter and/or an enhancer or other regulatory element of a gene selected from the group consisting of at least one of the genes identified in Table 3 and the ovarian and prostate tumor-specific genes disclosed herein operably linked to the coding region of a gene that is essential for replication of the vector, wherein the vector is adapted to replicate upon transfection into a diseased prostate cell. The promoter sequences can be discerned by searching the publicly available databases for BAC clones that cover the entire gene; thereafter, the cDNA for the gene can be compared to the genomic sequence. This will generally reveal the intron-exon boundaries and the start site of the gene. Once these are established, the promoter sequences can be inferred. Such vectors are able to selectively replicate in a cancer cell such as prostate cell, but not in a non-diseased cancer cell. The replication is conditional upon the presence in a diseased cancer cell, and not in a non-diseased cancer cell, of positive transcription factors that activate the promoter of the disclosed genes selected for each cancer, e.g., prostate cancer as identified in Table 3 or the prostate tumor-specific genes disclosed herein. It can also occur by the absence of transcription inhibiting factors that normally occur in a non-diseased cell, e.g., a prostate cell, and prevent transcription as a result of the promoter. Accordingly, when transcription occurs, it proceeds into the gene essential for replication, such that in the diseased cell, but not in non-diseased cell, replication of the vector and its attendant functions occur. With this vector, a diseased prostate cell, e.g., a prostate cancer cell, can be selectively treated, with minimal systemic toxicity.

In one embodiment, the viral vector is an adenoviral vector, which includes a coding region of a gene essential for replication of the vector, wherein the coding region is selected from the group consisting of E1a, E1b, E2 and E4 coding regions. The term “gene essential for replication” refers to a nucleic acid sequence whose transcription is required for the vector to replicate in the target cell. Preferably, the gene essential for replication is selected from the group consisting of the E1A and E1b coding sequences. Particularly preferred is the adenoviral E1A gene as the gene essential for replication. Methods for making such vectors are well known to the person of ordinary skill in the art as described, e.g., in Sambrook et al., in Molecular Cloning: A Laboratory Manual, Cold Spring Harbor, New York, 1989. The present invention provides novel viral vectors based on the oncolytic adenoviral vector strategy as described in U.S. Pat. No. 5,998,205, issued Dec. 7, 1999 to Hallenbeck et al. (the disclosure of which is hereby incorporated by reference in its entirety). In particular, oncolytic adenoviral vectors are disclosed in which expression of at least one adenoviral gene, which is essential for replication, is controlled by a tissue-specific promoter which is selectively transactivated in cancer cells. In one embodiment a tissue-specific promoter controls the expression of E1a. In a particularly preferred embodiment both the E1a and E4 genes are controlled by tumor-specific promoters. Methods for preparing tissue-specific replication vectors and their use in the treatment of cancer cells and other types of abnormal cells which are harmful or otherwise unwanted in vivo in a subject are described in detail, e.g., in U.S. Pat. No. 5,998,205. U.S. Pat. No. 5,698,443 describes adenoviral vectors, in which expression of a gene essential for replication is controlled by the PSA promoter/enhancer. Unlike the vectors of the present invention, however, the viral vectors described in this patent replicate in normal as well as diseased prostate cells, because PSA promoter/enhancer is active in normal cells as well as in diseased cells.

In a further embodiment, the invention provides nucleic acid constructs in which a heterologous gene product is expressed under the control of a promoter and/or an enhancer or other regulatory element of a gene selected from the group consisting of at least one of the genes identified in Table 3 and the ovarian and prostate tumor-specific genes disclosed herein. Such heterologous gene products are expressed when the construct is present in diseased cells, e.g., cancer cells, but not in normal, non-diseased cells. The heterologous gene product provides, in some embodiments, for the inhibition, prevention, or destruction of the growth of the diseased cell, e.g., a prostate cancer cell. The gene product can be RNA, e.g., antisense RNA or ribozyme, or proteins such as a cytokine, e.g., interleukin, interferon, or toxins such as diphtheria toxin, pseudomonas toxin, etc. The heterologous gene product can also be a negative selective marker such as cytosine deaminase. Such negative selective markers can interact with other agents to prevent, inhibit or destroy the growth of the diseased prostate or ovarian cells. U.S. Pat. No. 6,057,299, for example, describes the construction and use of nucleic acid constructs in which heterologous genes are placed under the control of a PSA enhancer. The nucleic acid constructs can be introduced into target cells by methods known to those of skill in the art. For example, one can incorporate the constructs into an appropriate vector such as those described above.

The vector of the present invention can be transfected into a helper cell line for viral replication and to generate infectious viral particles. Alternatively, transfection of the vector or other nucleic acid into a cancer cell can take place by electroporation, calcium phosphate precipitation, microinjection, or through liposomes, including proteoliposomes.

### EXAMPLE

The following example is offered to illustrate, but not to limit the present invention.

This Example describes the use of mRNA profiling of the ten most commonly fatal carcinomas, coupled with supervised machine learning algorithms, to identify subsets of genes whose expression is uniquely characteristic for each of these ten carcinomas. These genes were used to accurately predict the anatomic origin of 75 blinded carcinomas, including metastatic lesions, with up to 95% success rates. This study demonstrates the existence of subsets of genes whose transcription is characteristic of specific carcinomas, despite a wide-ranging appearance of the tumor cells, and illustrates the feasibility of predicting the anatomic site of tumor origin in the context of multiple diverse tumor classes.

A global approach to this problem is taken by identifying sets of genes whose expression is specific to carcinomas of the prostate, breast, colorectum, lung, ovary, gastroesophagus, pancreas, liver, kidney and bladder, which together account for 70% (˜400,000 cases) of all cancer-related deaths in the United States (see Greenlee et al., CA Cancer J. Clin., 50:7-33 (2000)). mRNA from 100 carefully dissected primary tumors is analyzed with oligonucleotide microarrays containing detectors for 12,533 genes to obtain quantitative measurements of gene transcription in each sample. The initial set of 100 primary carcinomas is comprised of 10 prostate adenocarcinomas, 9 bladder carcinomas (8 transitional cell carcinomas and 1 squamous cell carcinoma), 10 infilitrating ductal breast carcinomas, 10 colorectal adenocarcinomas, 11 gastroesophageal adenocarcinomas, 11 kidney carcinomas, 6 liver (hepatocellular) carcinomas, 10 serous papillary ovarian adenocarcinomas, 6 pancreatic carcinomas and 17 lung carcinomas (9 adenocarcinomas and 8 squamous cell carcinomas). Each specimen is assessed by frozen section examination, and areas rich in tumor are cut from the frozen blocks prior to RNA extraction. Care is taken to avoid non-neoplastic epithelium within the tumor samples. RNA extraction and hybridization is performed as described (see Lockhart et al., Nature Biotechnol., 14:1675-1680 (1996); and Wodicka et al., Nature Biotechnol., 15:1359-1367 (1997)), with the exception that the arrays are hybridized at 50° C. for 16-20 hours. GeneChip® hybridization data are processed and scaled as described (see Lockhart, supra; and Wodicka, supra)). Only those probe sets (9,198) are included whose maximum hybridization intensity (average difference (AD)) across all samples is >200; the other probe sets are excluded. All AD values <20, including negative AD values, are raised to a value of 20 and the data is log transformed. A complete description of all of the tumors used in this study and the primary hybridization data are available from our website (www.gnf.org/cancer/epican/tumors). Cancer classification schemes are then developed to identify specific sets of genes that could be used as classifiers to predict the anatomic origin of 75 unknown tumor samples. This provides a quantitative measure of the extent to which these genes are characteristic of an individual tumor type. Finally, individual genes are further characterized in the classifier sets to determine tissue versus tumor specific expression.

Initial analysis of gene expression in these tumors by simple hierarchical clustering reveals complex patterns of transcription. Although separate cancers of some anatomic sites, such as the prostate and kidney, can be readily separated based solely on the patterns of the most variably expressed genes, a striking degree of similarity is identified between cancers of the colorectum, stomach, bladder and lung, making histologic separation difficult. Therefore the process of multi-class prediction is divided into three components: a) filtering the large data set of gene expression (12,533 genes in 100 tumors, >1.25 million data-points) to exclude those genes that do not contribute to tumor distinction; b) ranking potentially predictive genes to identify the most accurate tumor-specific classifiers; and c) determining an optimal method by which these genes could be used to ‘vote’ for the likely class of a blinded tumor sample in the context of multiple tumor classes.

Genes in each tumor class that have the most significant probabilities of being differentially expressed relative to all other classes are first identified by a Wilcoxon rank test. For each of the 9,198 genes, a Wilcoxon rank score is calculated for the group with the highest mean expression versus samples from all other groups (implemented in Matlab v6.0). One hundred genes from each tumor class identified by this procedure are then subjected to a ‘prediction accuracy test’, in which each gene is individually evaluated for its ability to discriminate one tumor class from all other tumor classes using a supervised machine learning classifier. The 100 genes with the lowest p-values in each class (total 1,100 genes) are ranked based on their predictive accuracy for discriminating one tumor class versus all others using a support vector machine (SVM) classifier. Specifically, genes are ranked based on their LOOCV accuracy. In LOOCV for a given gene, we blind ourselves to one sample, trained an SVM using the remaining samples, and use the SVM to predict the class identity of the blinded sample (either cancer class X, or not cancer class X). This process is repeated for all samples in the training set, and an overall prediction accuracy is calculated for each gene. The SVM procedure (E. Dimitriadou, K. Homik, F. Leisch, D. Meyer, and A. Weingessel) is implemented in the software package R v1.2.2.4.

A voting scheme is developed based on calculating a ‘class distance’, by which to evaluate how molecularly related an unknown sample is to tumors of different classes. The voting scheme utilizes the 10 genes with the highest SVM/LOOCV accuracy from each class (110 total genes). For each class, a minimum SVM/LOOCV accuracy threshold is set such that at least ten genes passed; since in each class multiple genes have equivalent accuracy, 216 genes are selected from the 11 classes and were iteratively bootstrapped to obtain an equal number (10) of voting genes per class. For classifying an unknown sample, prediction scores are calculated using one set of 110 genes (calculated as described below), and final predictions are based on averaged scores over 50 iterations. Hybridization values for our 110-gene predictor set are compared to each sample in our training set. An L1 distance (sum of absolute differences) from the unknown sample to each training sample is calculated. The “class distance” is defined as the mean distance from the unknown sample to the members of that class in the training set. The class to which an unknown sample has the lowest class distance is the predicted identity.

A confidence score is also employed to estimate the strength of each prediction, and experimentally determines a confidence threshold that minimizes tumor misclassification. A Dixon test for outliers is employed to assign a confidence score to each prediction. The Dixon metric is calculated by sorting the vector of mean distances, where xᵢ<xᵢ₊₁, and computing the value D=(x₂−x₁)/(xₙ−x₁). A Dixon threshold of D=0.1 is empirically set as a conservative boundary for high confidence predictions. Empirically, it is determined that a small group of 110 genes, representing 10 genes per tumor class, most accurately predicts the origin of a blinded tumor sample (see Table 3).

Using these optimized parameters, the performance of the classification method is first assessed by predicting an anatomic site of origin for each of 100 tumors in the training set by cross-validation (see Table 1). Confident predictions are made for 94/100 (94%) of the samples, of which 92 (98%) are correct. The 6 unclassified cases do not pass the confidence threshold imposed on the experiment.

The classification scheme is then applied to an independent series of 75 cancer samples, which are blinded during training of the classifier. This group is comprised of tumors with histologies represented in our training set, including 12 metastatic lesions and many poorly differentiated tumors whose cellular features are not entirely indicative of their anatomic origin. Specifically, the set of 75 blinded tumor samples includes 63 primary tumors and 12 metastatic lesions. The primary tumor samples are 9 lung cancers (4 adenocarcinomas, 5 squamous cell carcinoma, 9 colorectal adenocarcinomas, 13 breast carcinomas, 14 prostate adenocarcinomas, 15 papillary serous ovarian carcinomas, 1 hepatocellular carcinoma and 2 gastroesophageal carcinomas). More detailed description of the ovarian and prostate cancer collection has been reported (see Welsh et al., Proc. Nat'l. Acad. Sci. USA, 98:1176-1181 (2002); and Welsh et al., Submittedfor publication (2001 Confident and accurate predictions for 64/75 (85%) tumors are made above the empirically set confidence threshold, including 9/12 (75%) metastatic cancers. None of these tumors samples are incorrectly classified. In the absence of the confidence threshold, an anatomic origin of 97/100 (97%) tumors is correctly predicted in the training set by cross-validation and 71/75 (95%) tumors in the blinded sample set, including 11/12 (92%) metastatic lesions.

TABLE 1
Prediction accuracy based on a 100-tumor training set

| | Number of | Dixon Confidence Threshold | | | No Confidence Threshold | |
|---|---|---|---|---|---|---|
| Tumor Set | tumors | Correct | Misclassified | No Call | Correct | Misclassified |
| Training set | 100 | 92 (92%) | 2 (2%) | 6 (6%) | 97 (97%) | 3 (3%) |
| (cross-validation) | | | | | | |
| Blinded set | 75 | 64 (85%) | 0 (0%) | 11 (15%) | 71 (95%) | 4 (5%) |

Two different groups of tumors were predicted using our classification method: 100 tumors comprising the training set (Training set) and a group of 75 tumors (Blinded set). Each sample in the training set was blinded and predicted in a cross-validation study. The blinded set contained samples not included in the training set, the identities of which were unknown during the training and optimization of the method.

Classification of tumors arising in certain anatomic sites is relatively straightforward because of the large number of unequivocal predictor genes (e.g., 19 genes with 100% predictive accuracy for prostate cancer). In contrast, prediction of other tumors, such as those of the lung, bladder or gastroesophagus, is more difficult because of the relative paucity of highly predictive classifier genes. The difficulty in selecting genes whose expression is specific to these cancers reflects a high degree of molecular relatedness, which we have observed upon initial analyses of tumor gene expression. For example, blinded gastroesophageal cancers that could not be predicted by our method are assigned as lung tumors (albeit with confidence scores close to zero; see Table 2). Analysis of the entire human transcriptome with these and other tumors may identify additional tumor-specific genes that would augment those identified here.

TABLE 2
Distribution of class predictions

| | | Predicted Class | | | | | | | | | | |
|---|---|---|---|---|---|---|---|---|---|---|---|---|
| | | PR | BL | BR | CO | GA | KI | LI | OV | PA | LA | LS |
| True | PR | 26 | | | | | | | | | | |
| identity | | (0.564) | | | | | | | | | | |
| of | BL | | 8 | | | | | | | | | |
| unknown | | | (0.343) | | | | | | | | | |
| sample | BR | | | 26 | | | | | | | | |
| | | | | (0.267) | | | | | | | | |
| | CO | | | | 23 | | | | | | | |
| | | | | | (0.279) | | | | | | | |
| | GA | | | | | 11 | | | | | 2 | |
| | | | | | | (0.187) | | | | | (0.044) | |
| | KI | | | | | | 11 | | | | | |
| | | | | | | | (0.502) | | | | | |
| | LI | | | 1 | | | | 5 | | | 1 | |
| | | | | (0.115) | | | | (0.523) | | | (0.041) | |
| | OV | | 1 | | | | | | 26 | | | |
| | | | (0.045) | | | | | | (0.317) | | | |
| | PA | | | | | | | | | 6 | | |
| | | | | | | | | | | (0.529) | | |
| | LA | | 1 | | | | | | | | 13 | |
| | | | (0.020) | | | | | | | | (0.275) | |
| | LS | | | 1 | | | | | | | | 13 |
| | | | | (0.138) | | | | | | | | (0.307) |

PR—prostate;
BL—bladder;
BR—breast;
CO—colorectal;
GA—gastroesophageal;
KI—kidney;
LI—liver;
OV—ovary;
PA—pancreas;
LA—lung adenocarcinomas;
LS—lung squamous cell carcinoma.

The genes that constitute the most accurate cancer class predictors (see Table 3) are divisible into two groups: those whose altered transcription is characteristic of specific neoplasms (termed ‘tumor-specific’), and those that are characteristic of the tissue in which they are normally expressed, rather than of the cancers that arise in these tissues (termed ‘tissue-specific’). On the basis of gene annotation alone, many well-described tumor-specific markers and targets are recognized. These include MUC-2 and A33 in colon cancers, the latter of which has been used as an immunotherapeutic target in advanced colorectal carcinomas (see Tschmelitsch et al., Cancer Res., 57:2181-2186 (1997)); mammaglobin-1 (MGB-1) and uroplakin II (UPII), which have been proposed as highly sensitive diagnostic markers for micro-metastatic breast and bladder cancers, respectively (see Ghossein et al., In vivo, 14:237-250 (2000); and Li et al., J. Urol., 162:931-935 (1999)); and thyroid transcription factor 1 (TFF-1), which has been proposed as a highly accurate marker for differential diagnosis of lung adenocarcinomas (see Reis-Filho et al., Pathol. Res. Pract., 196:835-840 (2000)). Examples of tissue-specific genes are kidney organic cation transporter, liver serum albumin and pancreatic lipase (see Table 3).

Interestingly, genes are also identified whose annotations suggest their expression in the stromal cells that surround epithelial tumors. In some cases, evidence is subsequently found of their over-expression in malignant epithelia (e.g., the fibroblast activation protein (FAP-α) in breast cancers (see Kelly et al., Mod. Pathol., 11:855-863 (1998)). In adenocarcinomas of the lung, genes are identified whose annotations indicate the presence of B-cells, T-cells, macrophages and neutrophils, reflecting a positive smoking history in these patients. With the exception of TTF-1, few of the genes identified in lung adenocarcinoma (see Table 3) are good classifiers.

Because of the inherent difficultly in using gene annotation alone to judge tumor-specific versus tissue-specific gene expression, it is sought to objectively ‘dissect’ predictor gene subsets into these different components. The levels of expression of 28 and highly-ranked predictor genes for ovarian and prostate cancers, respectively, are directly compared in normal and tumor tissues. The 29 prostate cancer-specific genes are chosen that are all >99% predictive of prostate cancer within the training set of 101 tumors, and 28 ovarian cancer-specific genes, which are at least 92% predictive of ovarian cancers. The expression levels of these genes in an expanded set of 24 ovarian and 24 prostate cancer samples are compared against 5 and 9 normal samples of ovary and prostate, respectively (see Welsh, supra; and Welsh, supra). Genes are ranked by an unpaired t-test and a measure of mean difference in expression levels. Differential expression is determined for genes whose expression is significantly different in normal and tumor tissues (p<0.01) and where the mean level of expression in tumor tissues is >3 times that in normal tissues. In ovarian cancers, 18/28 genes are significantly over-expressed in the tumors. Among this group of genes are protease M/neurosin/kallikrein 6 (hK6), which is a candidate serum marker for ovarian cancer (see Diamandis et al., Clin. Biochem., 33:579-583 (2000)), and mesothelin (CAKI), which is over-expressed in ovarian cancers and used as a specific target for a novel therapeutic immunotoxin (see Hassan et al., J. Immunother., 20:2902-2906 (2000)). Two G-protein coupled receptors, GPR39 and GPR64, are also identified which are important examples of potential tumor-specific therapeutic targets discovered by this approach. The 10 tissue-specific genes, which include the Wilm's tumor gene (WT-1), smad6 and Hox, most likely represent features of normal ovarian physiology. In prostate, significant cancer-specific up-regulation of 6 genes is observed, including the prostate-specific T-cell receptor gamma chain (TCRy. Two products of the TCRylocus are transcribed, of which the smaller transcript, translated as a 7 kDa protein termed TARP, is only expressed in the nucleus of prostate and breast cancer cells and is thought to be under the control of estrogens and androgens (see Wolfgang et al., Proc. Nat'l. Acad. Sci. USA, 15:9437-9442 (2000)). Over-expression of the calmodulin-dependent kinase 1 (CAMK1), testican, the multi-drug resistance gene, MRP4 (ABCC4) and a LIM domain protein is also found. To our knowledge, none of these have yet been reported as over-expressed in prostate cancers.

To evaluate whether the expression of these genes is tumor-specific in the context of gene expression in a whole array of human tissues, the expression of ovarian, prostate and other tumor-specific genes is analyzed in an expanded set of 46 normal human body tissues organs and cell lines. In ovarian cancers, for example, very few or no body tissues exhibit discernable expression of several of the ovarian cancer predictor genes, including CAK1 and hK6. Importantly, some of the genes with unknown function in several cancer-types are also highly tissue-specific, highlighting the potential of this method to identify novel, highly restricted tumor-specific genes for molecular intervention or diagnosis.

The increased tumor-specific transcription of a subset of these genes is further evaluated by analysis of over-expression of their protein products. For example, a polyclonal antibody specific to the Wilm's Tumor (WT) protein is used, whose transcript is highly-expressed in ovarian cancers, on tissue microarrays containing 229 carcinomas representing tumors from the 10 anatomic sites analyzed in the study. The tissue microarrays contain 0.6 mm cores from 265 different zinc formalin-fixed paraffin-embedded specimens and are constructed using a Tissue Microarrayer (Beecher Instruments, Silver Springs, Md.). Samples consist of 36 normal adult epithelial tissues and 229 carcinomas that include most of the tumors whose transcripts are profiled in the study. Ovarian cancers are profiled as previously described (see Welsh, supra) and 16 other independent serous papillary carcinomas of the ovary are included in the tissue microarrays. For immunohistochemistry on the tissue microarrays and on a whole tissue section of a normal ovary, the avidin-biotin immunoperoxidase method is performed. After slides had been placed in a citrate buffer and treated with microwave heat for 20 minutes, the polyclonal anti-WT antibody (C-19;1:100 dilution; Santa Cruz Biotechnology, Santa Cruz, Calif.) is applied for one hour at room temperature. Nuclear immunoreactivity is considered to represent true positivity. Immunostaining for WT protein is present in nuclei from 18/20 (90%) serous papillary carcinomas, while nuclear immunoreactivity is absent in the other 209 carcinomas. As expected from the analysis of tissue- and tumor-specific transcription in ovarian cancers, the normal serous lining epithelium of the ovary is also positive for WT protein. The results indicate that the derivation of antibodies to the products of tumor-specific genes described here can have clinical utility for early detection, predicting tumor origin, monitoring patients for tumor recurrence, and possibly antibody-based therapy. This potential is underscored by the identification of many known or predicted tumor diagnostic genes, such as MGB-1 in breast cancer, PSA and hK2 in prostate cancer, hK6 in ovarian cancer and uroplakins 1b and II in bladder carcinomas (see Table 3).

A striking conclusion from the data presented here is that subsets of genes with highly restricted, tumor-specific expression can be identified for as many as 11 distinct tumor classes, despite well-described tumor heterogeneity and obvious molecular similarities among many divergent tumor classes. The fact, that these gene subsets can be successfully used to predict the origin of a given tumor in a majority of cases, underscores how strongly characteristic these genes are for specific histopathological subtypes of cancer. In that regard, it is worth noting that using as few as 11 genes (i.e., one gene per tumor class), the anatomic origin can be predicted of up to 91% and 83% of the training and blinded tumor samples, respectively. These success rates indicate the broad applicability of these methods to other molecular classification problems, including identifying the molecular signatures of diverse toxicants or drug responses. The groups of predictor genes that are ‘tumor-specific,’ including some of those that are discussed above, are especially attractive because class distinction among tumors of diverse tissue origin inherently selects for tissue-specificity. These features are highly relevant for immunotherapeutic and chemical antagonism of gene function, as well as providing novel targets for gene therapy approaches for selective tumor cell destruction. Finally, these results demonstrate that one can construct custom DNA microarrays for a molecular classification of solid tumors, a resource that will augment traditional site-specific and histopathological classification schemes. This is particularly valuable for cases of metastases where the primary tumor origin is undetermined, estimated at 4% of all patients diagnosed with cancer (see Hillen, supra). The extension of these and other discriminant methods to identify molecular correlates with tumor grade, stage, response to therapy and outcome, further contribute to the optimal management of patients with cancer.

TABLE 3
Annotations of predictor genes

| Cancer | Affy | | | LOOCV |
|---|---|---|---|---|
| Type | Probe ID | Name | Description | Accuracy |
| PR | 251_at | CAMK1 | calcium/calmodulin-dependent protein kinase I | 100 |
| PR | 37812_at | KIAA0293 | KIAA0293 protein | 100 |
| PR | 40794_at | KLK3 | kallikrein 3, (prostate specific antigen) | 100 |
| PR | 41721_at | KLK2 | kallikrein 2, prostatic | 100 |
| PR | 41468_at | TRG@ | T cell receptor gamma locus | 100 |
| PR | 1513_at | LBX1 | transcription factor similar to D. melanogaster | 100 |
| | | | homeodomain protein lady bird late | |
| PR | 41172_at | LOC51109 | CGI-82 protein | 100 |
| PR | 32200_at | ACPP | acid phosphatase, prostate | 100 |
| PR | 217_at | KLK2 | kallikrein 2, prostatic | 100 |
| PR | 1514_g_at | none | Antigen \|TIGR == HG2261-HT2352 | 100 |
| PR | 40297_at | STEAP | six transmembrane epithelial antigen of the prostate | 100 |
| PR | 617_at | ACPP | acid phosphatase, prostate | 100 |
| PR | 35778_at | KIF5C | kinesin family member 5C | 100 |
| PR | 1662_r_at | none | Antigen \|TIGR == HG2261-HT2351 | 100 |
| PR | 263_g_at | AMD1 | S-adenosylmethionine decarboxylase 1 | 100 |
| PR | 36685_at | AMD1 | S-adenosylmethionine decarboxylase 1 | 100 |
| PR | 1661_i_at | none | Antigen \|TIGR == HG2261-HT2351 | 100 |
| PR | 40060_r_at | LIM | LIM protein (similar to rat protein kinase C-binding enigma) | 100 |
| PR | 1805_g_at | KLK3 | kallikrein 3, (prostate specific antigen) | 100 |
| BL | 32448_at | UPK2 | uroplakin 2 | 96 |
| BL | 36628_at | RALBP1 | ralA binding protein 1 | 96 |
| BL | 36555_at | SNCG | synuclein, gamma (breast cancer-specific protein 1) | 96 |
| BL | 36571_at | TOP2B | topoisomerase (DNA) II beta (180 kD) | 96 |
| BL | 38457_at | TNNI2 | troponin I, skeletal, fast | 95 |
| BL | 1490_at | MYCL1 | v-myc avian myelocytomatosis viral oncogene | 95 |
| | | | homolog 1, lung carcinoma derived | |
| BL | 37104_at | PPARG | peroxisome proliferative activated receptor, gamma | 95 |
| BL | 39939_at | COL4A6 | collagen, type IV, alpha 6 | 95 |
| BL | 32527_at | APM2 | adipose specific 2 | 95 |
| BL | 35402_at | DR6 | death receptor 6 | 95 |
| BL | 41111_at | BCAT2 | branched chain aminotransferase 2, mitochondrial | 95 |
| BL | 32382_at | UPK1B | uroplakin 1B | 95 |
| BR | 41348_at | IRX5 | iroquois homeobox protein 5 | 96 |
| BR | 33848_r_at | CDKN1B | cyclin-dependent kinase inhibitor 1B (p27, Kip1) | 95 |
| BR | 33878_at | FLJ13612 | hypothetical protein FLJ13612 | 93 |
| BR | 36329_at | MGB1 | mammaglobin 1 | 92 |
| BR | 34778_at | none | Homo sapiens cDNA FLJ12280 fis, clone MAMMA1001744 | 92 |
| BR | 37142_at | GFRA1 | GDNF family receptor alpha 1 | 92 |
| BR | 686_s_at | none | Homo sapiens endogenous retrovirus HERV-K104 long | 92 |
| | | | terminal repeat, complete sequence; and Gag protein | |
| | | | (gag) and envelope protein (env) genes, complete cds | |
| BR | 39945_at | FAP | fibroblast activation protein, alpha | 92 |
| BR | 40161_at | COMP | cartilage oligomeric matrix protein (pseudoachondroplasia, | 92 |
| | | | epiphyseal dysplasia 1, multiple) | |
| BR | 1177_at | FLJ12443 | hypothetical protein FLJ12443 | 91 |
| BR | 40046_r_at | C18ORF1 | chromosome 18 open reading frame 1 | 91 |
| BR | 40162_s_at | COMP | cartilage oligomeric matrix protein (pseudoachondroplasia, | 91 |
| | | | epiphyseal dysplasia 1, multiple) | |
| CO | 170_at | CDX2 | caudal type homeo box transcription factor 2 | 97 |
| CO | 169_at | CDX1 | caudal type homeo box transcription factor 1 | 95 |
| CO | 37423_at | SLC12A2 | solute carrier family 12 (sodium/potassium/chloride | 95 |
| | | | transporters), member 2 | |
| CO | 38884_at | CLCA1 | chloride channel, calcium activated, family member 1 | 94 |
| CO | 37875_at | GPA33 | glycoprotein A33 (transmembrane) | 94 |
| CO | 32972_at | NOX1 | NADPH oxidase 1 | 94 |
| CO | 896_at | MUC2 | mucin 2, intestinal/tracheal | 94 |
| CO | 40736_at | CDH17 | cadherin 17, LI cadherin (liver-intestine) | 93 |
| CO | 41073_at | GPR49 | G protein-coupled receptor 49 | 93 |
| CO | 37415_at | ATP10B | ATPase, Class V, type 10B | 93 |
| CO | 41728_at | KIAA0152 | KIAA0152 gene product | 93 |
| CO | 1582_at | CEACAM5 | carcinoembryonic antigen-related cell adhesion molecule 5 | 93 |
| CO | 38739_at | ETS2 | v-ets avian erythroblastosis virus E26 oncogene homolog 2 | 93 |
| GA | 40957_at | KIAA0160 | KIAA0160 protein | 96 |
| GA | 302_at | AHCYL1 | S-adenosylhomocysteine hydrolase-like 1 | 95 |
| GA | 31574_i_at | none | Human HL14 gene encoding beta-galactoside- | 94 |
| | | | binding lectin, 3′ end, clone 2 | |
| GA | 710_at | P4HB | procollagen-proline, 2-oxoglutarate 4-dioxygenase (proline | 93 |
| | | | 4-hydroxylase), beta polypeptide (protein disulfide | |
| | | | isomerase; thyroid hormone binding protein p 55) | |
| GA | 34595_at | MYHL | myosin, heavy polypeptide-like (110 kD) | 93 |
| GA | 38116_at | KIAA0101 | KIAA0101 gene product | 93 |
| GA | 36015_at | CORT | cortistatin | 93 |
| GA | 31575_f_at | none | Human HL14 gene encoding beta-galactoside- | 93 |
| | | | binding lectin, 3′ end, clone 2 | |
| GA | 39253_s_at | RALA | v-ral simian leukemia viral oncogene homolog A (ras related) | 92 |
| GA | 34851_at | STK6 | serine/threonine kinase 6 | 92 |
| GA | 40451_at | none | AL080203: Homo sapiens mRNA; cDNA DKFZp434F222 (from clone DKFZp434F222)/ | 92 |
| | | | cds = (0 \|GenBank == AL080203 | |
| GA | 34491_at | OASL | 2′-5′-oligoadenylate synthetase-like | 92 |
| KI | 33534_at | ESM1 | endothelial cell-specific molecule 1 | 100 |
| KI | 35220_at | ENPEP | glutamyl aminopeptidase (aminopeptidase A) | 100 |
| KI | 35243_at | PCTK3 | PCTAIRE protein kinase 3 | 100 |
| KI | 39654_at | ASPA | aspartoacylase (aminoacylase 2, Canavan disease) | 99 |
| KI | 35867_at | SLC22A2 | solute carrier family 22 (organic cation transporter), member 2 | 99 |
| KI | 1951_at | ANGPT2 | angiopoietin 2 | 99 |
| KI | 39260_at | SLC16A4 | solute carrier family 16 (monocarboxylic acid transporters), member 4 | 99 |
| KI | 34777_at | ADM | adrenomedullin | 99 |
| KI | 40954_at | FXYD2 | FXYD domain-containing ion transport regulator 2 | 99 |
| KI | 36668_at | DIA1 | diaphorase (NADH) (cytochrome b-5 reductase) | 99 |
| LI | 37816_at | C5 | complement component 5 | 100 |
| LI | 32771_at | GC | group-specific component (vitamin D binding | 99 |
| | | | protein) | |
| LI | 37175_at | SERPINC1 | serine (or cysteine) proteinase inhibitor, clade C | 99 |
| | | | (antithrombin), member 1 | |
| LI | 37235_g_at | KNG | kininogen | 99 |
| LI | 37236_at | none | M11437: Human kininogen gene/cds = (0 \|GenBank == M11437 | 99 |
| LI | 36342_r_at | HFL3 | H factor (complement)-like 3 | 99 |
| LI | 36657_at | APOC2 | apolipoprotein C-II | 99 |
| LI | 40311_at | TFR2 | transferrin receptor 2 | 99 |
| LI | 33701_at | PAH | phenylalanine hydroxylase | 99 |
| LI | 36341_s_at | HFL3 | H factor (complement)-like 3 | 99 |
| LI | 39763_at | HPX | hemopexin | 99 |
| LI | 37202_at | F2 | coagulation factor II (thrombin) | 99 |
| LI | 261_s_at | APOB | apolipoprotein B (including Ag(x) antigen) | 99 |
| LI | 33377_at | VTN | vitronectin (serum spreading factor, somatomedin | 99 |
| | | | B, complement S-protein) | |
| LI | 36584_at | ITIH2 | inter-alpha (globulin) inhibitor, H2 polypeptide | 99 |
| LI | 35332_at | APOB | apolipoprotein B (including Ag(x) antigen) | 99 |
| LI | 1232_s_at | IGFBP1 | insulin-like growth factor binding protein 1 | 99 |
| OV | 32625_at | NPR1 | natriuretic peptide receptor A/guanylate cyclase A | 99 |
| | | | (atrionatriuretic peptide receptor A) | |
| OV | 1500_at | WT1 | Wilms tumor 1 | 99 |
| OV | 38201_at | BCAT1 | branched chain aminotransferase 1, cytosolic | 98 |
| OV | 40763_at | MEIS1 | Meis1 (mouse) homolog | 98 |
| OV | 35277_at | SPON1 | spondin 1, (f-spondin) extracellular matrix protein | 98 |
| OV | 40401_at | LOC55816 | hypothetical protein | 97 |
| OV | 34194_at | none | Homo sapiens mRNA; cDNA DKFZp564B076 | 97 |
| | | | (from clone DKFZp564B076) | |
| OV | 32959_at | none | M25809: Human endomembrane proton pump | 96 |
| | | | subunit mRNA \|GenBank == M25809 | |
| OV | 37554_at | KLK6 | kallikrein 6 (neurosin, zyme) | 96 |
| OV | 35226_at | EYA2 | eyes absent (Drosophila) homolog 2 | 95 |
| OV | 38749_at | GPR39 | G protein-coupled receptor 39 | 95 |
| OV | 32838_at | none | S67247: smooth muscle myosin heavy chain | 95 |
| | | | isoform SMemb [human \|GenBank == S67247 | |
| OV | 1955_s_at | MADH6 | MAD (mothers against decapentaplegic, Drosophila) homolog 6 | 95 |
| PA | 39177_r_at | CEL | carboxyl ester lipase (bile salt-stimulated lipase) | 100 |
| PA | 41238_s_at | ELA3 | elastase 3, pancreatic (protease E) | 100 |
| PA | 35594_at | PNLIPRP2 | pancreatic lipase-related protein 2 | 100 |
| PA | 31482_at | CELL | carboxyl ester lipase-like (bile salt-stimulated lipase-like) | 99 |
| PA | 386_g_at | CTRL | chymotrypsin-like | 99 |
| PA | 36141_at | CTRB1 | chymotrypsinogen B1 | 99 |
| PA | 39176_f_at | CEL | carboxyl ester lipase (bile salt-stimulated lipase) | 99 |
| PA | 39726_at | GCG | glucagon | 99 |
| PA | 34941_at | CLPS | colipase, pancreatic | 99 |
| PA | 40714_at | CTRC | chymotrypsin C (caldecrin) | 99 |
| PA | 31483_g_at | CEL | carboxyl ester lipase (bile salt-stimulated lipase) | 99 |
| PA | 40043_at | PRSS3 | protease, serine, 3 (trypsin 3) | 99 |
| PA | 40748_at | CPA2 | carboxypeptidase A2 (pancreatic) | 99 |
| PA | 912_s_at | PLA2G1B | phospholipase A2, group IB (pancreas) | 99 |
| PA | 32796_f_at | PRSS2 | protease, serine, 2 (trypsin 2) | 99 |
| PA | 41369_at | PNLIP | pancreatic lipase | 99 |
| PA | 34309_at | CPA1 | carboxypeptidase A1 (pancreatic) | 99 |
| PA | 38936_at | ELA1 | elastase 1, pancreatic | 99 |
| PA | 40204_at | CTRL | chymotrypsin-like | 99 |
| LA | 33754_at | TITF1 | thyroid transcription factor 1 | 98 |
| LA | 40928_at | DKFZP564A122 | DKFZP564A122 protein | 94 |
| LA | 40749_at | MS4A2 | membrane-spanning 4-domains, subfamily A, member 2 | 94 |
| | | | (Fc fragment of IgE, high affinity I, receptor for; beta polypeptide) | |
| LA | 130_s_at | TITF1 | thyroid transcription factor 1 | 94 |
| LA | 37634_at | PAEP | progestagen-associated endometrial protein (placental protein | 93 |
| | | | 14, pregnancy-associated endometrial alpha-2-globulin, | |
| | | | alpha uterine protein) | |
| LA | 34876_at | none | U65090: Human carboxypeptidase D mRNA | 93 |
| | | | \|GenBank == U65090 | |
| LA | 33383_f_at | ASAHL | N-acylsphingosine amidohydrolase (acid ceramidase)-like | 93 |
| LA | 32116_at | LAK-4P | expressed in activated T/LAK lymphocytes | 92 |
| LA | 31901_at | KCNAB2 | potassium voltage-gated channel, shaker-related | 91 |
| | | | subfamily, beta member 2 | |
| LA | 32640_at | ICAM1 | intercellular adhesion molecule 1 (CD54), human | 91 |
| | | | rhinovirus receptor | |
| LA | 40019_at | EVI2B | ecotropic viral integration site 2B | 91 |
| LA | 37148_at | LILRB3 | leukocyte immunoglobulin-like receptor, subfamily B | 91 |
| | | | (with TM and ITIM domains), member 3 | |
| LA | 31457_at | FOXD2 | forkhead box D2 | 91 |
| LA | 38332_at | MGC11256 | hypothetical protein MGC11256 | 91 |
| LA | 40520_g_at | PTPRC | protein tyrosine phosphatase, receptor type, C | 91 |
| LA | 32793_at | TRB@ | T cell receptor beta locus | 91 |
| LA | 41165_g_at | none | X67301: H. sapiens mRNA for IgM heavy chain | 91 |
| | | | constant region (Ab63)/cds = (0 \|GenBank == X67301 | |
| LA | 1478_at | ITK | IL2-inducible T-cell kinase | 91 |
| LA | 41164_at | none | X67301: H. sapiens mRNA for IgM heavy chain | 91 |
| | | | constant region (Ab63)/cds = (0 \|GenBank == X67301 | |
| LA | 33956_at | MD-2 | MD-2 protein | 91 |
| LA | 32616_at | LYN | v-yes-1 Yamaguchi sarcoma viral related oncogene homolog | 91 |
| LA | 37402_at | RNASE1 | ribonuclease, RNase A family, 1 (pancreatic) | 91 |
| LA | 36878_f_at | HLA-DQB1 | major histocompatibility complex, class II, DQ beta 1 | 91 |
| LA | 38894_g_at | NCF4 | neutrophil cytosolic factor 4 (40 kD) | 91 |
| LA | 40518_at | PTPRC | protein tyrosine phosphatase, receptor type, C | 91 |
| LA | 32632_g_at | GBA | glucosidase, beta; acid (includes glucosylceramidase) | 91 |
| LA | 36773_f_at | HLA-DQB1 | major histocompatibility complex, class II, DQ beta 1 | 91 |
| LA | 37351_at | UP | uridine phosphorylase | 91 |
| LA | 31680_at | TOP1 | topoisomerase (DNA) I | 91 |
| LA | 34874_at | NTE | neuropathy target esterase | 91 |
| LA | 37637_at | RGS3 | regulator of G-protein signalling 3 | 91 |
| LA | 37344_at | HLA-DMA | major histocompatibility complex, class II, DM alpha | 91 |
| LA | 1633_g_at | PIM2 | pim-2 oncogene | 91 |
| LA | 1461_at | NFKBIA | nuclear factor of kappa light polypeptide gene | 91 |
| | | | enhancer in B-cells inhibitor, alpha | |
| LA | 402_s_at | ICAM3 | intercellular adhesion molecule 3 | 91 |
| LA | 37218_at | BTG3 | BTG family, member 3 | 91 |
| LA | 195_s_at | CASP4 | caspase 4, apoptosis-related cysteine protease | 91 |
| LA | 37170_at | none | AB015331: Homo sapiens HRIHFB2017 mRNA | 91 |
| | | | \|GenBank == AB015331 | |
| LA | 37411_at | ACAP1 | KIAA0050 gene product | 91 |
| LA | 40476_s_at | ILF1 | interleukin enhancer binding factor 1 | 91 |
| LA | 36440_at | none | Human pre TCR alpha mRNA, partial cds | 91 |
| LA | 1062_g_at | IL10RA | interleukin 10 receptor, alpha | 91 |
| LA | 32558_at | PIAS3 | protein inhibitor of activated STAT3 | 91 |
| LA | 32715_at | VAMP8 | vesicle-associated membrane protein 8 (endobrevin) | 91 |
| LA | 1173_g_at | none | Spermidine/Spermine N1-Acetyltransferase | 91 |
| | | | \|TIGR == HG172-HT3924 | |
| LA | 40406_at | MST1 | macrophage stimulating 1 (hepatocyte growth factor-like) | 91 |
| LA | 1665_s_at | none | Homo sapiens CDA02 mRNA, complete cds | 91 |
| LA | 32193_at | none | Homo sapiens clone 23785 mRNA sequence | 91 |
| LA | 39134_at | TOM1 | target of myb1 (chicken) homolog | 91 |
| LA | 35132_at | MYO1E | myosin IE | 91 |
| LA | 39296_at | FBLN1 | fibulin 1 | 91 |
| LA | 568_at | PRKACA | protein kinase, cAMP-dependent, catalytic, alpha | 91 |
| LA | 38378_at | CD53 | CD53 antigen | 91 |
| LA | 33273_f_at | IGLJ3 | immunoglobulin lambda joining 3 | 91 |
| LA | 31526_f_at | USP6 | ubiquitin specific protease 6 (Tre-2 oncogene) | 91 |
| LA | 33274_f_at | IGLJ3 | immunoglobulin lambda joining 3 | 91 |
| LA | 37864_s_at | none | Y14737: Homo sapiens mRNA for immunoglobulin lambda | 91 |
| | | | heavy chain/cds = (65 \|GenBank == Y14737 | |
| LA | 33354_at | SMURF2 | E3 ubiquitin ligase SMURF2 | 91 |
| LA | 36493_at | LSP1 | lymphocyte-specific protein 1 | 91 |
| LA | 1427_g_at | SLA | Src-like-adapter | 91 |
| LA | 32091_at | KIAA0446 | KIAA0446 gene product | 91 |
| LA | 34677_f_at | none | Homo sapiens mRNA for TL132 | 91 |
| LA | 39649_at | ARHGAP4 | Rho GTPase activating protein 4 | 91 |
| LA | 31820_at | HCLS1 | hematopoietic cell-specific Lyn substrate 1 | 91 |
| LA | 927_s_at | MUC1 | mucin 1, transmembrane | 91 |
| LA | 41827_f_at | IGLL1 | immunoglobulin lambda-like polypeptide 1 | 91 |
| LA | 41091_at | FALZ | fetal Alzheimer antigen | 91 |
| LA | 38762_at | RNAHP | RNA helicase-related protein | 91 |
| LA | 1729_at | TRADD | TNFRSF1A-associated via death domain | 91 |
| LA | 38869_at | KIAA1069 | KIAA1069 protein | 91 |
| LA | 37352_at | SP100 | nuclear antigen Sp100 | 91 |
| LA | 35248_at | SLC19A2 | solute carrier family 19 (thiamine transporter), member 2 | 91 |
| LA | 40635_at | FLOT1 | flotillin 1 | 91 |
| LA | 37975_at | CYBB | cytochrome b-245, beta polypeptide (chronic granulomatous disease) | 91 |
| LA | 34304_s_at | SAT | spermidine/spermine N1-acetyltransferase | 91 |
| LA | 38666_at | PSCD1 | pleckstrin homology, Sec7 and coiled/coil domains 1(cytohesin 1) | 91 |
| LS | 39015_f_at | KRT6A | keratin 6A | 98 |
| LS | 39016_r_at | KRT6A | keratin 6A | 97 |
| LS | 33529_at | ADH7 | alcohol dehydrogenase 7 (class IV), mu or sigma polypeptide | 96 |
| LS | 1560_g_at | PAK2 | p21 (CDKN1A)-activated kinase 2 | 96 |
| LS | 33693_at | DSG3 | desmoglein 3 (pemphigus vulgaris antigen) | 96 |
| LS | 31791_at | TP63 | tumor protein 63 kDa with strong homology to p53 | 96 |
| LS | 32563_at | ATP1B3 | ATPase, Na+/K+ transporting, beta 3 polypeptide | 96 |
| LS | 33108_i_at | SOX2 | SRY (sex determining region Y)-box 2 | 95 |
| LS | 32380_at | PKP1 | plakophilin 1 (ectodermal dysplasia/skin fragility syndrome) | 95 |
| LS | 1932_at | ABCC5 | ATP-binding cassette, sub-family C (CFTR/MRP), member 5 | 95 |
| LS | 36457_at | GMPS | guanine monophosphate synthetase | 95 |
| LS | 39581_at | CSTA | cystatin A (stefin A) | 95 |
| LS | 1933_g_at | ABCC5 | ATP-binding cassette, sub-family C (CFTR/MRP), member 5 | 95 |

Of the 12,533 probe sets interrogated by the oligonucleotide arrays, the 216 genes shown here were selected as predictors in the classification method.
PR—prostate;
BL—bladder;
BR—breast;
CO—colorectal;
GA—gastroesophageal;
KI—kidney;
LI—liver;
OV—ovary;
PA—pancreas;
LA—lung adenocarcinomas;
LS—lung squamous cell carcinoma.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

We claim:

1. A method for identifying an origin of a tumor, the method comprising detecting in a tumor sample an expression level of at least two genes, each of which genes is diagnostic for a different tumor class as identified in Table 3, wherein an elevated level expression for a gene indicates that the tumor originated from the tumor class for which the gene is diagnostic.

2. The method or claim 1, wherein the tumor is of prostate cancer, breast cancer, colorectal cancer, lung adenocarcinoma, lung squamous cell carcinoma, ovarian cancer, gastroesophageal cancer, pancreatic cancer, liver cancer, kidney cancer or bladder cancer origin.

3. The method of claim 1, wherein an expression level is determined for at least three genes, each of which genes is diagnostic for a different tumor class as identified in Table 3.

4. The method of claim 1, wherein an expression level is determined for at least two genes that are both diagnostic for a single tumor class as identified in Table 3.

5. The method of claim 1, wherein an expression level is determined for at least two genes that are both diagnostic for a first tumor class as identified in Table 3, and at least two genes that are both diagnostic for a second tumor class as identified in Table 3.

6. The method of claim 1, wherein an expression level is determined for at least three genes in each of two or more tumor classes as identified in Table 3.

7. The method of claim 1, wherein an expression level is determined for one or more genes in each of at least ten tumor classes identified in Table 3.

8. The method of claim 1, wherein the expression level is elevated compared to expression level of the gene in a non-cancer control sample.

9. The method of claim 1, wherein the expression level is elevated compared to expression of the gene in a control sample obtained from a tumor of a different tumor class.

10. The method of claim 1, wherein the expression level of a gene is determined by detecting the level of expression of an mRNA transcribed from the gene.

11. The method of claim 10, wherein the level of expression of mRNA is detected by techniques selected from the group consisting of northern blot analysis, reverse transcriptase PCR, real time quantitative PCR and hybridization to an oligonucleotide array.

12. The method of claim 1, wherein the expression level of a gene is determined by detecting the level of expression of a protein encoded by the gene.

13. The method of claim 12, wherein the level of expression of the protein is detected through western blotting or an array by utilizing a labeled probe specific for the protein.

14. The method of claim 13, wherein the probe is an antibody.

15. The method of claim 14, wherein the antibody is a monoclonal antibody.

16. The method of claim 1, wherein the tumor is a metastatic lesion or a primary tumor.

17. A method for identifying an origin of a tumor, the method comprising:
a) providing a predictor set that comprises expression levels for two or more genes, each of which is diagnostic for a different tumor class as identified in Table 3;
b) detecting in a tumor sample an expression level of at one gene that is diagnostic for a tumor class as identified in Table 3; and
c) calculating a vector distance from the expression level obtained from the tumor sample to each of the expression levels of the predictor set,
wherein the shortest vector distance indicates the origin of the tumor.

18. The method of claim 17, wherein the predictor set comprises expression levels for at least three genes, each of which genes is diagnostic for a different tumor class as identified in Table 3.

19. The method of claim 17, wherein the predictor set comprises expression levels for at least two genes that are both diagnostic for a single tumor class as identified in Table 3.

20. The method of claim 17, wherein the predictor set comprises expression levels for at least two genes that are both diagnostic for a first tumor class as identified in Table 3, and at least two genes that are both diagnostic for a second tumor class as identified in Table 3.

21. The method of claim 17, wherein the predictor set comprises expression levels for at least three genes in each of two or more tumor classes as identified in Table 3.

22. The method of claim 17, wherein the predictor set comprises expression levels for one or more genes in each of at least ten tumor classes identified in Table 3.

23. The method of claim 17, wherein the expression level of a gene in the tumor sample is determined by detecting the level of expression of an mRNA transcribed from the gene.

24. The method of claim 17, wherein the expression level of a gene in the tumor sample is determined by detecting the level of expression of a protein encoded by the gene.

25. The method of claim 17, wherein the tumor sample is obtained from a metastatic lesion or a primary tumor.

26. The method of claim 17, wherein the Dixon threshold for the shortest vector distance is 0.5 or less.

27. The method of claim 26, wherein the Dixon threshold for the shortest vector distance is 0.1 or less.
